(19) United States
(12) Patent Application Publication
(10) Pub. No.: US 2003/0036532 A1
(43) Pub. Date: Feb. 20, 2003

(54) Use of 2-phenylene diamine derivatives for the treatment of infections

(76) Inventors: Hassan Jomaa, Giessen (DE); Martin Schlitzer, Amoneburg-Rossdorf (DE); Jochen Wiesner, Giessen (DE)

Correspondence Address: WILLIAM COLLARD, COLLARD & ROE, P.C., 1077 NORTHERN BOULEVARD, ROSLYN, NY, 11576, US

(21) Appl. No.: 10/204,683

(22) PCT Filed: Feb. 22, 2001
(86) PCT No.: PCT/EP01/02016

(30) Foreign Application Priority Data
Feb. 24, 2000 (DE) 100 08 522.9

(51) Int. Cl. A61K 31/675; A61K 31/66; A61K 31/44; A01N 43/40; A61K 31/27; A61K 31/40

(52) U.S. Cl. 514/79; 514/114; 514/352; 514/426; 514/521; 514/523; 514/585; 514/595; 514/603; 514/619; 514/567; 514/485

(57) ABSTRACT

The invention relates to the use of compounds of formula (I), wherein n=0-3; R¹, R²=H, alkyl, aryl, heteroaryl, acyl; R³=H, halogen, alkyl, aryl, heteroaryl, arylalkyl, acyl, CN, NO₂, R⁴—X—; R⁴=H, alkyl, aryl, heteroaryl, aralkyl, acyl; X=NH, O, S, SO₂, NHSO₂, OSO₂, and A, B, C=organic groups. The inventive compounds are used for the prophylaxis and the therapeutic treatment of infectious processes, especially of infectious processes caused by parasites. The invention further relates to medicaments that contain the inventive compounds.

The present invention concerns the use of 2-phenylene diamine derivatives for prophylactic and therapeutic treatment of infections. It especially concerns the treatment of infections caused by parasites.

Malaria is one of the main reasons for the high mortality in tropic regions with 300 to 500 million clinical cases and 1.5 to 2.5 million deaths a year. Since the parasites' resistance against conventional antimalaria agents is growing alarmingly, new agents are urgently needed.

Thus, it is an object of the present invention, to provide for new agents for combating parasitic infections, especially malaria.

This object is achieved in a completely surprising manner by the group of substances 2-phenylene diamine derivatives defined in claim 1 gelöst. This group of substances not only demonstrates an anti-infectious effect against uni and multicellular parasites, but also against viruses, bacteria and fungi. Within the meaning of the present invention unicellular parasites mean protozoa according to the narrow definition of parasitology.

The compounds used according to the present invention have already been proposed for the therapy of cancer, as disclosed in the simultaneously pending application of the present applicant.

The 2-phenylene diamine derivatives correspond to general formula (I):

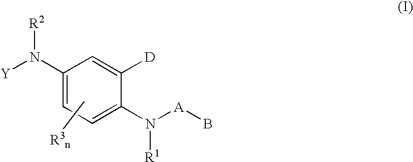

wherein

n=0-3;

R¹, R²=H, C₁₋₂₆-alkyl, aryl, heteroaryl, C₁₋₂₆-acyl;

R³=H, halogen, C₁₋₂₆-alkyl, aryl, heteroaryl, ar-C₁₋₂₆-alkyl, C₁₋₂₆-acyl, CN, NO₂, R⁴—X—;

with R⁴=H, C₁₋₂₆-alkyl, aryl, heteroaryl, ar-C₁₋₂₆-alkyl, C₁₋₂₆-acyl;

X=NH, O, S, SO₂, NHSO₂, OSO₂,

A=CH₂, CHR⁵, CR⁵R⁶, CO, CS, CONR⁴, CSNR⁴, SO₂, PO₂,

R⁵, R⁶=independently of each other C₁₋₂₆-alkyl, aryl, heteroaryl, ar-C₁₋₂₆-alkyl, CN, NO₂, COR⁷,

R⁷=H, C₁₋₂₆-alkyl, aryl, ar-C₁₋₂₆-alkyl, C₁₋₂₆-alkoxy, aryloxy, ar-C₁₋₂₆-alkoxy, NR⁸R⁹,

R⁸, R⁹=independently of each other H, C₁₋₂₆-alkyl, aryl, ar-C₁₋₂₆-alkyl, heteroaryl,

B=C₁₋₂₆-alkyl, aryl, heteroaryl, arylcarbonyl, saturated heterocycles, substituted alkyl having 1-4 chain members, wherein the substituents may be C₁₋₉-alkyl, aryl, heteroaryl, halogen, ═O, OH, NH₂, NH—CO—R¹⁰, NH—SO₂—R¹⁰, COOR¹¹, CO—NR¹²R¹³, CS—NR¹⁴R¹⁵, SO₂OR¹⁶, SO₂NR¹⁷R¹⁸, NH—CO—OR¹⁹, NH—CO—NR²OR²¹, NHCSNR²²R²³,

R¹⁰-R₂₃=independently of each other H, C₁₋₂₆-alkyl, aryl, ar-C₁₋₂₆-alkyl, heteroaryl,

D=H, C₁₋₂₆-alkyl, aryl, heteroaryl, ar-C₁₋₂₆-alkyl, —Y—R²⁴, halogen, NO₂, CN, NH—CO—R²⁵, NH—SO₂—R²⁶, NH—CO—OR²⁷, NH—CO—NR²⁸R²⁹, NH—CS—NR³OR³¹,

Y=O, NH, S, CO, CS, SO₂, COO, CONR³¹, CSNR³², SO₂NR³³,

R²⁴-R³³=independently of each other H, C₁₋₂₆-alkyl, aryl, heteroaryl, ar-C₁₋₂₆-alkyl, and

Y=a group selected from

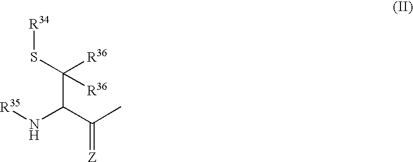

wherein

Z=O, S or two hydrogen atoms,

R³⁴=H, C₁₋₂₆-alkyl, aryl, ar-C₁₋₂₆-alkyl, COOR³⁷, arylsulfonyl,

R³⁵=H, C₁₋₂₆-acyl, COOR³⁸,

R³⁶=independently of each other H, C₁₋₂₆-alkyl,

R³⁷, R³⁸=independently of each other C₁₋₂₆-alkyl, aryl, ar-C₁₋₂₆-alkyl, or

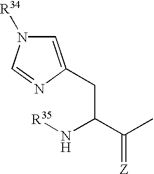

wherein

Z=O, S or two hydrogen atoms,

R³⁴=H, C₁₋₂₆-alkyl, aryl, ar-C₁₋₂₆-alkyl, COOR³⁷, arylsulfonyl,

R³⁵=H, C₁₋₂₆-acyl, COOR³⁸

R³⁷, R³⁸=independently of each other C₁₋₂₆-alkyl, aryl, ar-C₁₋₂₆-alkyl, or

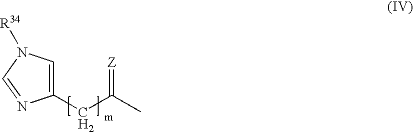

wherein

Z=O, S or two hydrogen atoms,

m=0-3

R³⁴=H, C₁₋₂₆-alkyl, aryl, ar-C₁₋₂₆-alkyl, COOR³⁷, aryl sulfonyl,

R³⁷=C₁₋₂₆-alkyl, aryl, ar-C₁₋₂₆-alkyl, or

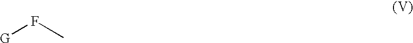

wherein

F=CH₂, CO, CS, SO₂,

G=COOR³⁹, CONHOH, CONR⁴⁰R⁴¹, CSNR⁴²R⁴³, C₁₋₂₆-alkyl- or alkyl substituted by aryl and having 1-3 chain members or alkyl having 1-3 chain members, which supports a substituent at the terminal C-atom the substituent selected from COOR⁴⁴, CONHOH, CONR⁴⁵R⁴⁶, CSNR⁴⁷R⁴⁸, SR⁴⁹, SOR⁵⁰, SO₂R⁵¹, SO₂NR⁵²R⁵³, PO(OR⁵⁴)OR⁵⁵, PO(OR⁵⁶)NR⁵⁷₂, OSO₂R⁵⁸, O(PO)OR⁵⁹, NHSO₂R⁶⁰, NHPO₂R⁶¹, NHCOR⁶², NHCSR⁶³, NHCONR⁶⁴R⁶⁵, NHCSNR⁶⁶R⁶⁷, —S(NH)₂—R⁶⁸ or NH(C═NR⁷⁰)NHR⁶⁹, furthermore aryl or heteroaryl,

R³⁹-R⁶⁹=independently of each other H, C₁₋₂₆-alkyl, aryl,

R⁷⁰=CONH₂, SO₂NH₂, or is

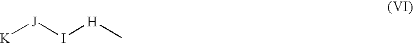

wherein

H=CH₂, CO, CS, CHR⁷¹, CR⁷²R⁷³, SO₂, SO, PO₂,

I=C₁₋₂₆-alkylene, C₁₋₂₆-alkylene, in which one methylene group is replaced by O, S, or NR⁷⁷, or C₂₋₂₆-alkenylene, these alkylene or alkenylene radicals being unsubstituted or substituted by aryl, heteroaryl, halogen, OH, CN, C₁₋₉-alkyloxy, aryloxy, COOR⁷⁴, CONR⁷⁵R⁷⁶, NR⁷⁷R⁷⁸, NH(C═NR⁷⁰)NHR⁶⁹, SR⁷⁹, SO₂R⁸⁰; furthermore C₃₋₈-cycloalkylene, unsubstituted or substituted by aryl, heteroaryl, halogen, OH, CN, C₁₋₉-alkyloxy, aryloxy, COOR⁷⁴, CONR⁷⁵R⁷⁶, NR⁷⁷R⁷⁸, SR⁷⁹, SO₂R⁸⁰; C₃₋₈-cycloalkylene, wherein the alkylene chain is interrupted by O, S or NR⁷⁷, arylene, unsubstituted or substituted by aryl, heteroaryl, halogen, OH, CN, C₁₋₂₆ alkoxy, aryloxy, COOR⁷⁴, CONR⁷⁵R⁷⁶, NR⁷⁷R⁷⁸, SR⁷⁹, SO₂R⁸⁰,

R⁷¹-R⁸⁰=independently of each other H, C₁₋₂₆-alkyl, aryl, ar-C₁₋₂₆-alkyl, heteroaryl,

J=a bonding or CO, COO, CONR⁸¹, CS, CSNR⁸², SO₂, S(NH)₂, SO(NH), SO₂₀, SO²NR⁸³, PO(OR⁸⁴), PO(OR⁸⁵)NR⁸⁶, NR⁸⁷CO, NR⁸⁸CS, NR⁸⁹SO₂, OSO₂,NR⁹⁰PO(OR⁸⁴), OPO(OR⁹¹), PO(OR⁸⁴)O, NR⁹²CONR⁹³, NR⁹⁴CSNR⁹⁵, NR⁹⁶SO₂NR⁹⁷, NR⁹⁸C(NR⁹⁹)NR¹⁰⁰,

R⁸¹-R¹⁰⁰=independently of each other H, C₁₋₂₆-alkyl, aryl, ar-C₁₋₂₆-alkyl, heteroaryl,

R⁹⁹=H, C₁₋₂₆-alkyl, aryl, ar-C₁₋₂₆-alkyl, heteroaryl, CONR¹⁰¹R¹⁰², CSNR¹⁰³R¹⁰⁴, SO₂NR¹⁰⁵R¹⁰⁶

R¹⁰¹-R¹⁰⁶=independently of each other H, C₁₋₂₆-alkyl, aryl, ar-C₁₋₂₆-alkyl, heteroaryl,

K=branched or unbranched C₁₋₂₃-alkyl, branched or unbranched C₁₁₋₂₃-alkenyl being unsubstituted or substituted by aryl or heteroaryl, C₁₁₋₂₃-alkinyl, aryl, heteroaryl, ar-C₁₁₋₂₆-alkyl,

wherein aryl, heteroaryl and ar-C₁₋₂₆-alkyl are substituted by further aryl-, heteroaryl- and/or ar-C₁₋₂₆-alkyl radicals,

and the salts thereof.

For the compounds which are especially remarkable because of their activity the following definitions are valid:

n=0-3,

R¹, R² and R³=H,

A=CO, SO₂,

B=phenyl, benzyl, phenethyl, 4-chlorophenylmethyl, 4-bromophenylmethyl, 4-nitrophenylmethyl, 4-trifluoromethylphenylmethyl, 4-methylphenylmethyl, 1-(4-methyl-1-piperazinyl)-1-(4-trifluoromethylphenyl)methyl, 1-(4-methyl-1-piperazinyl)-1-phenylmethyl, 4-phenylphenyl-methyl, 1-naphthylmethyl, 2-naphthylmethyl, benzoyl, 2,4,4-trimethylpentyl, 2-carboxyethyl, 3-carboxypropyl,

D=benzoyl,

Y=

a radical of formula (II),

wherein

Z=O,

R³⁴=H, benzyloxycarbonyl, trityl,

R³⁵=H, benzyloxycarbonyl, tert.-butyloxycarbonyl,

R³⁶=H,

or a radical of formula (III),

wherein

Z=O,

R³⁴=H, tosyl, benzyl, 4-nitrobenzyl, 4-cyanobenzyl, benzyloxycarbonyl,

R³⁵=H, benzyloxycarbonyl, tert.-butyloxycarbonyl,

or a radical of formula (IV),

wherein

Z=O,

m=0-3,

R³⁴=H, tosyl, benzyl, 4-nitrobenzyl, 4-cyanobenzyl, benzyloxycarbonyl,

or a radical of formula (V),

wherein

F=CO, CH₂, SO₂,

G=COOH, COOMe, CONHOH, CH₂—COOH, CH₂COOMe, CH₂CONHOH, CH₂CONH—(C₁₄-C₂₀)-alkyl, CH₂CH₂COOH, CH₂CH₂COOMe, CH₂CH₂CONHOH, CH₂CH₂CONH—(C₁₄-C₂₀)-alkyl, CH₂CH₂CH₂COOH, CH₂CH₂CH₂COOMe, CH₂CH₂CH₂CONHOH, CH₂CH₂CH₂CONH—(C₁₄-C₂₀)-alkyl, phenyl, naphthyl, pyrridyl, fluorenyl, anthracenyl,

or a radical of formula (VI),

wherein

H=CO, SO₂,

I=methylene, 1,2-ethylene, 1,3-trimethylene, 1,4-tetramethylene, —CH₂—S—CH₂—, —CH₂—O—CH₂—, —CH₂—NH—CH₂—, 1,2-ethenylene, 1,1-ethylene, prop-1-en-1,3-ylene, prop-2-en-1,3-ylene, benzylene, 2-phenyl-1,1-ethylene, carboxymethylene, amino carbonyl methylene, 2-Carboxy-1,1-ethylene, 2-aminocarbonyl-1,1-ethylene, 3-carboxy-1,1-propylene, 3-aminocarbonyl-1,1-propylene, 2-methyl-1,1-propylene, 3-methyl-1,1-butylene, 2-methyl-1,1-butylene, pyrrolidine connected via N1 and C2, 1,2-phenylene, 1,3-phenylene, 1,2-naphthylene, 1,3-naphthylene, 1,1-cyclopentylene, 1,1-cyclohexylene, 1,2-cyclohexylene,

J=a bonding, CO, CS, SO₂, PO(OMe), PO(OH), CONH, CSNH, SO₂NH, PO(OH)O, PO(OH)NH, PO(OMe)O, PO(OMe)NH,

K=(C₁₃-C₁₉)-alkyl, (C₁₃-C₁₉)-alkenyl, 4-benzyloxystyryl, 4-styrylstyryl, 4-phenylstyryl, 4-cyanostyryl, 4-nitrostyryl, phenyl, 4-biphenylyl, 4-nitrophenyl, 4-cyanophenyl, 4-methylsulfonylphenyl, 4-methoxyphenyl, 4-bromophenyl, 4-chlorophenyl, 4-trifluoromethylphenyl, 4-Formylphenyl, 4-methoxycarbonylphenyl, 4-(1,1-dicyano-2-vinyl)phenyl, 4-aminophenyl, 4-ethylphenyl, 4-Isopropylphenyl, 4-tert.-butylphenyl, 4-ethoxyphenyl, 4-propoxyphenyl, 4-butoxyphenyl, 4-benzyloxyphenyl, 3,4-bis-(benzyloxy)phenyl 3-phenoxyphenyl, 4-styrylphenyl 1-naphthyl, 2-naphthyl, 2-fluorenyl, 2-(2-phenylthiazol-4-yl), 5-(4-nitrophenyl)thiazo-4-yl) 5-(4-nitrophenyl)-furan-2-yl), 5-(3-nitrophenyl)furan-2-yl), 5-(2-nitrophenyl)furan-2-yl), 5-(4-bromophenyl)furan-2-yl), 5-(4-chlorophenyl)furan-2-yl), 5-(3-trifluoromethylphenyl)-furan-2-yl), 5-(4-trifluoromethylphenyl)furan-2-yl), 5-(2-chloro-3-trifluoromethylphenyl)-furan-2-yl) 3,4-methylenedioxyphenyl, 1-acetylindol-3-yl, 4′-nitrobiphenylyl, 5-(4-bromophenyl)thiophen-2-yl), 5-(4-methylphenyl)furan-2-yl), 5-(4-methoxyphenyl)furan-2-yl), 5-bromothiophen-2-yl, 5-bromofuran-2-yl, 4-pyridyl, 3-pyridyl, 2-pyridyl, Chinolin-2-yl, 1-naphthylvinyl, 2-naphthylvinyl, 2-fluorenyl-vinyl, 2-(2-phenylthiazol-4-yl)vinyl, 2-[5-(4-nitrophenyl)furan-2-yl)vinyl, 2-[5-(4-acetoxy-methylphenyl)furan-2-yl)vinyl, 2-[5-(3-trifluoromethylphenyl)furan-2-yl)vinyl, 4-benzyloxy-styryl, 3,4-dibenzyloxystyryl, 3-methoxy-4-(4-nitrobenzyloxy)styryl, 2-methylindol-3-ylvinyl, 1-acetylindol-3-ylvinyl, 3,4-methylenedioxystyryl, 4-(1,1 dicyano-2-vinyl)styryl and the salts thereof.

The compounds of formula (I) are especially preferred, for which it is valid:

n=0-3,

R¹, R²=H

R³=H,

A=CO, SO₂,

B=phenyl, benzyl, phenethyl, 4-chlorophenylmethyl, 4-methylphenylmethyl, 1-(4-methyl-1-piperazinyl)-1-(4-trifluoromethylphenyl)methyl, 1-(4-methyl-1-piperazinyl)-1-phenylmethyl, 4-bromophenylmethyl, 4-nitrophenylmethyl, 4-trifluoromethylphenylmethyl, 4-phenylphenylmethyl, 1-naphthylmethyl, 2-naphthylmethyl, benzoyl, 2,4,4-trimethylpentyl, 2-Carboxyethyl, 3-Carboxypropyl,

D=benzoyl,

Y=

a radical of formula (IV)

wherein

Z=O,

m=0-3,

R³⁴=H, benzyl, 4-nitrobenzyl, 4-cyanobenzyl,

or a radical of formula (V)

wherein

F=CO, CH₂, SO₂,

G=CH₂CH₂CONH—(C₁₄-C₁₈)-alkyl, CH₂CH₂CH₂CONH—(C₁₄-C₁₈)-alkyl, phenyl, naphthyl, pyridyl, fluorenyl, anthracenyl

or a radical of formula (VI)

H=CO,

I=methylene, 1,2-ethylene, 1,3-trimethylene, 1,4-tetramethylene, —CH₂—S—CH₂—, —CH₂—O—CH₂—, —CH₂—NH—CH₂—, 1,2-ethenylene, 1,1-ethylene, prop-1-en-1,3-ylene, prop-2-en-1,3-ylene, benzylene, 2-phenyl-1,1-ethylene, 2-methyl-1,1-propylene, 3-methyl-1,1-butylene, 2-methyl-1,1-butylene, pyrrolidine connected via N1 and C2,

J=PO(OMe), PO(OH), CONH, CSNH, SO₂NH, PO(OH)O, PO(OH)NH, PO(OMe)O, PO(OMe)NH,

K=(C₁₃-C₁₉)-alkyl, (C₁₃-C₁₉)-alkenyl, 4-benzyloxystyryl, 4-phenylstyryl, 4-cyanostyryl, 4-nitrostyryl, phenyl, 4-biphenylyl, 4-nitrophenyl, 4-cyanophenyl, 4-methoxyphenyl, 1-naphthylvinyl, 2-naphthylvinyl, 2-fluorenylvinyl, 2-(2-phenylthiazol-4-yl)vinyl, 2-[5-(4-nitrophenyl)furan-2-yl)vinyl, 2-[5-(4-acetoxymethylphenyl)furan-2-yl)vinyl, 2-[5-(3-trifluoromethylphenyl)furan-2-yl)vinyl, 3,4-dibenzyloxystyryl, 3-methoxy-4-(4-nitrobenzyloxy)styryl, 3,4-methylenedioxystyryl, 4-(1,1dicyano-2-vinyl)styryl and the salts thereof.

The single compounds corresponding to the following formulae of structure are especially preferred:

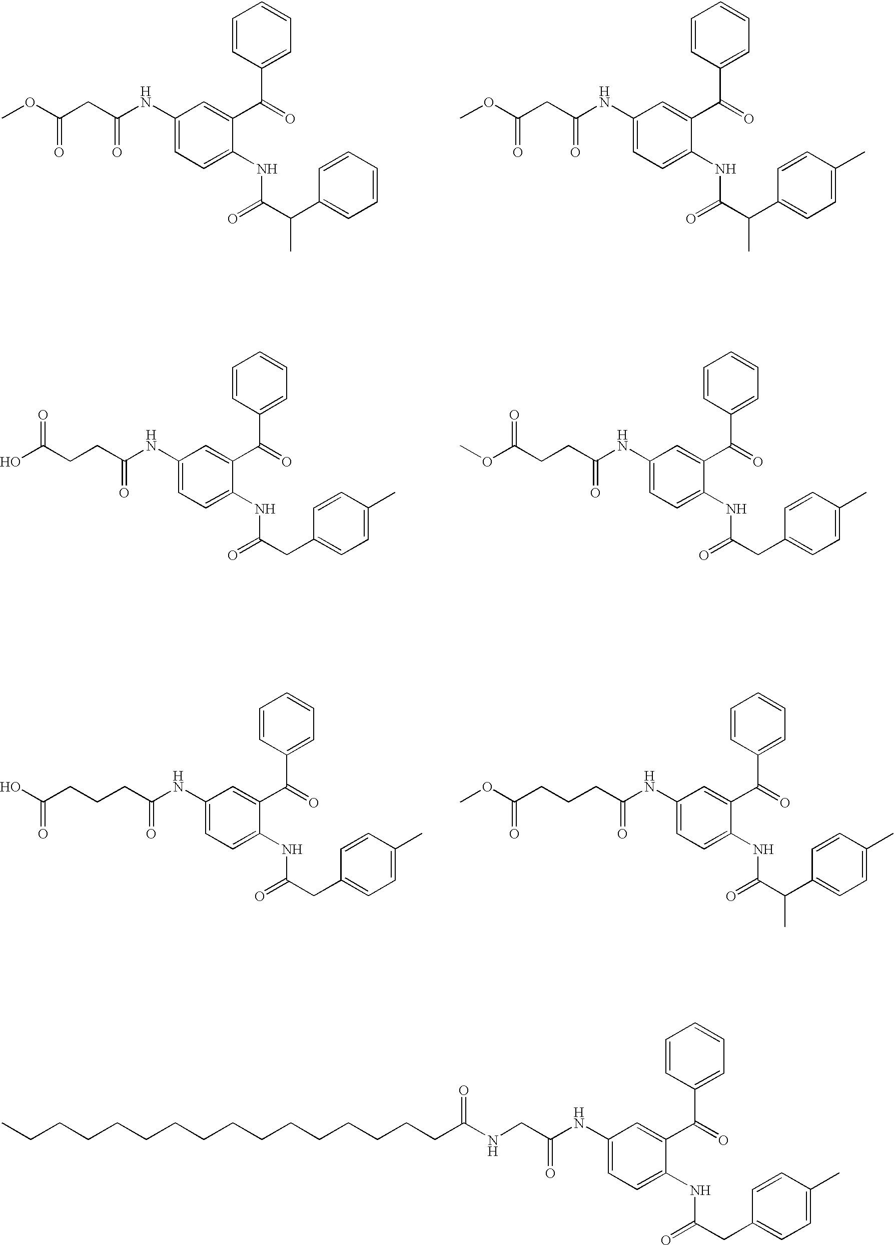

Out of these compounds N-[3-[3-benzoyl-4-[2-(4-methylphenyl)-acetyl]amino]-phenylamino]-4-phenyl cinnamon acid amide and N-[2-[3-benzoyl-4-[[2-(4-methylphenyl)acetyl]-amino]phenyl]-4-benzyloxy cinnamon acid amide are especially preferred.

In the above and in the following formulae and definitions acyl especially represents alkanoyl as well as alkanoyl substituted by aryl. Acyl groups having 1 to 5 carbon atoms are preferred. Alkyl, also in derived terms, such as alkoxy, alkylene, alkenyl and alkinyl is straight-chain or branched-chain, contains, as far as not stated otherwise, especially 1 to 8 C-atoms and is un-substituted or substituted by e.g. CN, NH₂, NO₂, COOH, CONH₂ and alkoxycarbonyl. aryl mainly represents phenyl, substituted by e.g. halogen, alkyl, trifluoromethyl, cyano, aryl, alkoxy, hydroxy, benzyloxy, phenyl, styryl, acyl, NO₂, COOH, alkylsulfonyl, SO₂NH₂ substituted phenyl, naphthyl, naphthyl substituted by e.g. halogen, alkyl, aryl, alkoxy, acyl, NO₂, COOH, SO₂NH₂, furthermore e.g. also fluorenyl and anthracenyl. The same meanings are valid for arylenes accordingly. Heteroaryl is e.g. a six membered aromatic substance or a five membered aromatic substance containing 1 to 4 heteroatoms, whereby heteroatoms are understood to be nitrogen, oxygen and sulfur, e.g. pyridyl, furanyl, thiazolyl, furthermore e.g. also indolyl. Heteroaryl is unsubstituted or substituted like aryl and especially also substituted by aryl. Aralkyl represents alkyl being mono- or polysubstituted by aryl, preferably mono- to trisubstituted. Cycloalkylene, wherein the alkylene chain is interrupted by O, S or NR77, is e.g. pyrrolidine connected via N1 and C2, halogen stands for fluor, chlor, brom and iod.

The compounds are in particular suited for the therapeutic and prophylactic treatment of infections in humans and animals caused by viruses, bacteria, and fungi, unicellular and multicellular parasites. They exhibit strong cytotoxic activity against uni- and multicellular parasites, in particular against the causative organisms of malaria and sleeping sickness

The compounds are preferably usable against unicellular parasites (protozoa), in particular against pathogens of malaria and the sleeping sickness as well as Chagas' disease, toxoplasmosis, amoebic dysentery, leishmaniasis, trichomoniasis, balantidiasis, cryptosporidiasis, sarcocystosis, acanthamebiasis, naegleriasis, coccidiosis, giardiasis and lambliosis.

Therefore, they are particularly suitable as malaria prophylactics and as prophylactics of sleeping sickness as well as the Chagas' disease, toxoplasmosis, amoebic dysentery, leishmaniasis, trichomoniasis, pneumocystosis, balantidiasis, cryptosporidiasis, sarcocystosis, acanthamebiasis, naegleriasis, coccidiosis, giardiasis and lambliosis.

The compounds according to the invention which generally include pharmaceutically acceptable salts or else compounds which upon application provide the compounds according to the invention as metabolic products or decomposition products, also called “prodrugs” may all be prepared for administration like known anti-infectious agents in any suitable manner (mixed with non-toxic pharmaceutically acceptable carriers).

The pharmaceutically effective preparations may be prepared in the form of pharmaceutical preparations in dispensing units. This means that the preparations can be present in the form of individual parts, for example tablets, dragees, capsules, pills, suppositories and ampoules, the active ingredient content of which corresponds to a fraction or a multiple of a single dose.

The dispensing units can, for example, contain 1, 2, 3 or 4 single doses or ½, ⅓ or ¼ of a single dose. A single dose preferably contains the quantity of active ingredient which is admistered during one application and which usually corresponds to a whole, a half or third of a quarter of a daily dose.

Non-toxic, inert pharmaceutically suitable carriers are understood to mean solid, semi-solid or liquid diluents, fillers and formulation auxiliary agents of all kinds.

Tablets, dragees, capsules, pills, granules, suppositories, solutions, suspensions and emulsions, pastes, ointments, gels, creams, lotions, powders and sprays are mentioned as preferred pharmaceutical preparations. Tablets, dragees, capsules, pills and granules may contain in addition to the conventional excipients the active ingredient, such as (a) fillers and diluents, for example starches, lactose, cane sugar, glucose, mannitol and silicic acid, (b) binders, for example carboxymethylcellulosis, alginate, gelatine, polyvinylpyrrolidone, (c) moisture-retaining agents, for example glycerol, (d) dispersing agents, for example agar-agar, calcium carbonate and sodium carbonate, (e) solution retarders, for example paraffin and (f) resorption accelerators, for example quaternary ammonium compounds, (g) wetting agents, for example cetyl alcohol, glycerol monostearate, (h) adsorption agents, e.g. kaolin and betonite and (i) lubricants, for example talcum, calcium and magnesium stearate and solid polyethylene glycol or mixtures of the substances listed under (a) to (i).

The tablets, dragées, capsules, pills and granules may be provided with the conventional coatings and casings optionally comprising opaquing agents and may also be put together so that they release the active ingredient or active ingredients only or preferably in a specific part of the intestinal optionally with sustain release, wherein polymer substances and waxes for example may be used as embedding compounds.

The active ingredient or the active ingredients may optionally also be present in microencapsulated form with one or more of the above mentioned excipients.

In addition to the active ingredient or the active ingredients suppositories may also contain the conventional water soluble or water insoluble excipients, for example polyethylene glycols, fats, for example cacoa fat and higher esters (for example C₁₄-alcohol with C₁₆-fatty acid) or mixtures of these substances.

In addition to the active ingredients ointments, pastes, creams and gels may contain the conventional excipients, for example animal and vegetable fats, waxes, paraffins, starch, tragacanth, cellulose derivative, polyethylene glycols, silicones, bentonites, silicic acid, talcum and zinc oxide or mixtures of these substances.

In addition to the active ingredients powders and sprays may contain the conventional excipients, for example lactose, talcum, silicic acid, aluminium hydroxide, calcium silicate and polyamide powder or mixtures of these substances. Sprays may additionally contain the conventional blowing agents, for example chlorofluorohydrocarbons.

In addition to the active ingredients solutions and emulsions may contain the conventional excipients such as solvents, solubilisers and emulgators, for example water, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular cotton seed oil, peanut oil, corn oil, olive oil, castor oil and sesame oil, glycerol, glycerol formal, tetrahydrofurfuryl alcohol, polyethyleneglycols and fatty acid esters of sorbitan or mixtures of these substances.

The solutions and emulsions may also be present in sterile and blood isotonic form for parenteral application.

In addition to the active ingredients suspensions may contain conventional excipients such as liquid diluents, for example water, ethyl alcohol, propylene glycol, suspending agents, for example ethoxylated isostearyl alcohols, polyoxyethylene sorbitol and sorbitan esters, microcrystalline cellulose, aluminum metahydroxide, bentonite, agar-agar and tragacanth or mixtures of these substances.

The active agents of formula (I) should be present in the above listed pharmaceutical preparations preferably in a concentration of approximately 0.1 to 99.5% by weight, preferably of approximately 0.5 to 95% by weight of the total mixture.

The above-mentioned formulations can also contain dyes, preservatives and odour and flavour improving additives, for example peppermint oil and eucalyptus oil and sweeteners, for example saccharine.

The compounds may be used with the hitherto described substances having antibacterial, antiviral, antimycotic and antiparasitic properties. Such substances in particular include compounds which have already been used in therapeutic applications or are still used. Substances which are suitable for this purpose are in particular those listed in the Red List or in Simon/Stille, Antibiokia-Therapie in Klinik und Praxis, 9th edition, 1998, Schatauer Verlag, or on the Internet at http://www.customs.treas.gov/imp-exp/rulings/harmoniz/hrm 129.html. The derivatives may in particular be present with penicillins, benzylpenicillin (penicillin G), phenoxypenicillins, isoxazolylpenicillins, aminopenicillins, ampicillin, amoxicillin, bacampicillin, carboxypenicillin, ticarcillin, temocillin, acylaminopenicillins, aziocillin, mezlocillin, piperacillin, apalcillin, mecillinam, cephalosporins, cefazolin group, cefuroxime group, cefoxitin group, cefoxitin, cefotetan, cefmetazole, latamoxef, flomoxef, cefotaxime group, cefozidime, ceftazidime group, ceftazidime, cefpirome, cefepime, conventional cephalosporins, cefsulodin, cefoperazone, oral cephalosporins of the cephalexin group, loracarbef, cefprozil, new broad-spectrum oral cephalosporins, cefixime, cefpodoxime-proxetil, cefuroxime-axetil, cefetamet, cefotiam-hexetil, cefdinir, ceftibuten, other B-lactam antibiotics, carbapenem, imipenem/cilastatin, meropenem, biapenem, aztreonam, B-lactamase inhibitors, clavulanic acid/amoxicillin, clavulanic acid/ticarcillin, sulbactam/ampicillin, tazobactam/piperacillin, tetracyclines, oxytetracycline, rolitetracycline, doxycycline, minocycline, chloramphenicol, aminoglycosides, gentamicin, tobramycin, netilmicin, amikacin, spectinomycin, macrolides, erythromycin, clarithromycin, roxithromycin, azithromycin, dirithromycin, spiramycin, josamycin, lincosamides, clindamycin, fusidic acid, glycopeptide antibiotics, vancomycin, teicoplanin, pristinamycin derivatives, fosfomycin, antimicrobial folic acid antagonists, sulfonamides, co-trimoxazole, trimethoprim, other diaminopyrimidine-sulfonamide combinations, nitrofurans, nitrofurantoin, nitrofurazone, gyrase inhibitors (quinolones), norfloxacin, ciprofloxacin, ofloxacin, sparfloxacin, enoxacin, fleroxacin, pefloxacin, lomefloxacin, Bay Y3118, nitroimidazoles, antimycobacterial agents, isoniazid, rifampicin, rifabutin, ethambutol, pyrazinamide, streptomycin, capreomycin, prothionamide, terizidone, dapsone, clofazimine, topical antibiotics, bacitracin, tyrothricin, polymyxins, neomycin, kanamycin, paromomycin, mupirocin, antiviral agents, acyclovir, ganciclovir, azidothymidine, didanosine, zaicitabine, thiacytidine, stavudine, ribavirin, idoxuridine, trifluridine, foscamet, amantadine, interferons, tibol derivatives, proteinase inhibitors, antimycotics, polyenes, amphotericin B, nystatin, natarnycin, azoles, azoles for septic therapy, miconazole, ketoconazole, itraconazole, fluconazole, UK-109,496, azoles for topical use, clotnimazole, econazole, isoconazole, oxiconazole, bifonazole, flucytosine, griseofulvin, ciclopirox olamine, tolnafnate, naftifine, terbinafine, amorolfine, anthraquinones, betulinic acid, semianthraquinones, xanthones, naphthoquinones, arylamino alcohols, quinine, quinidines, mefloquine, halofantrine, chloroquine, amodiaquine, acridine, benzonaphthyridine, mepacrine, pyronaridine, dapsone, sulfonamides, sulfadoxine, sulfalenes, trimethoprim, proguanil, chlorproguanil, diaminopyrimidines, pyrimethamine, primaquine, aminoquinolines, WR 238,605, tetracycline, doxycycline, clindamycin, norfloxacin, ciprofloxacin, ofloxacin, artemisinin, dihydroartemisinin, lOb artemether, arteether, atresunate, atovaquone, suramin, melarsoprol, nifurtimox, stibogluconate sodium, pentamidine, amphotericin B, metronidazole, clioquinol, mebendazole, niclosamide, praziquantel, pyrantel, tiabendazole, diethylcarbamazine, ivermectin, bithionol, oxamniquine, metrifonate, piperazine, embonate.

Furthermore the compounds may be present in the pharmaceutical preparations in combination with sulfonamide, sulfadoxin, artemisinin, atovaquon, chinin, chloroquine, hydroxychloroquine, mefloquin, halofantrin, pyrimethamine, armesin, tetracycline, doxycyclin, proguanil, metronidazol, praziquantil, niclosamide, mebendazol, pyrantel, tiabendazole, diethylcarbazin, piperazin, pyrivinum, metrifonate, oxamniquin, bithionol or suramin or several of these substances.

The above listed pharmaceutical preparations are produced in the conventional manner by known methods, for example by mixing the active ingredient or active ingredients with the excipient or excipients.

The above-mentioned preparations can be used in humans and animals either orally, rectally, parentally, (intravenously, intramuscularly, subcutaneously), intracisternally, intravaginally, intraperitoneally, topically (powder, ointment, drops) and for the treatment of infections in cavities, orifices. Suitable preparations are injection solutions, solutions and suspensions for oral treatment, gels, infusions, emulsions, ointments or drops. Ophthalmological and dermatogical formulations, silver and other salts, eardrops, eye ointments, powders or solutions can be used for topical treatment. With animals the absorption can occur via the food or drinking water in suitable formulations. Furthermore gels, powders, tablets, sustain release tablets, premixes, concentrates, granules, pellets, tablets, boli, capsules, aerosoles, sprays, inhalers can be used with humans and animals. The compounds according to the invention can furthermore be incorporated into other carrier materials such as, for example, plastic materials (plastic chains for topical treatment), collagen or bone cement.

In general it has proved advantageous both in human and veterinary medicine to administer the active ingredient or ingredients of formula (I) in total quantities of approximately 0.05 to approximately 600, preferably 0.5 to 200 mg/kg body weight per 24 hours, optionally in the form of several individual doses in order to achieve the desired results. An individual dose contains the active ingredient or ingredients preferably in quantities of approximately 1 to approximately 200, in particular 1 to 60 mg/kg body weight. It may, however, be necessary to deviate from the above-mentioned dosages and this is dependent on the nature and the body weight of the patient to be treated, the nature and the severity of the disease, the nature and the method and the application of the pharmaceutical compositions as well as the time scale or interval within the administration takes place.

Thus in some cases it may be sufficient to get by with less than the above mentioned quantity of active ingredient, whilst in other cases the above-stated quantity of active ingredient must be exceeded. The person skilled in the art may determine the optimum dosage and method of application of the active ingredient in each case by virtue of his expert experience.

The compounds according to the invention may be administered in animals in the conventional concentrations and preparations together with the feed or feed preparations or the drinking water.

The compounds according to the invention are prepared in principally known manner, for example by

(a) 2-acyl-4-nitroanilin is acylated with a suitable acyl chloride in inert solvent at higher temperature,

(b) 4-nitroanilide obtained under (a) is reduced by tin dichloride or palladium/hydrogen to the respective amino compound,

(c) the amino compound obtained under (b)is acylated with suitable substituted carboxylic acids, substituted carboxylic acid anhydrides or N-substituted amino acids, whereby N-acylamino acids in general are activated via the mixed anhydride method; and

(d) if in (c) protected amino acid derivatives are used, any. existing protective groups are split off by use of standard techniques of peptide chemistry.

The preparation of the compounds is shown in schemes 1-4 by example:

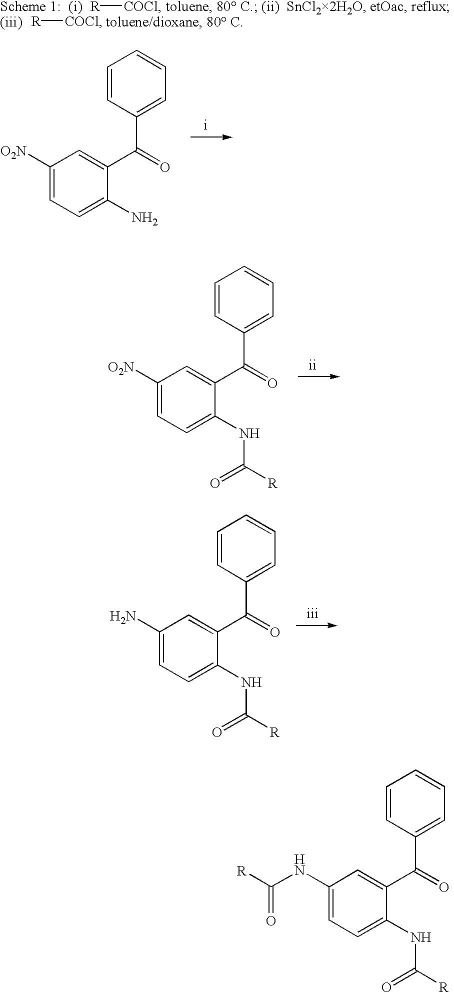

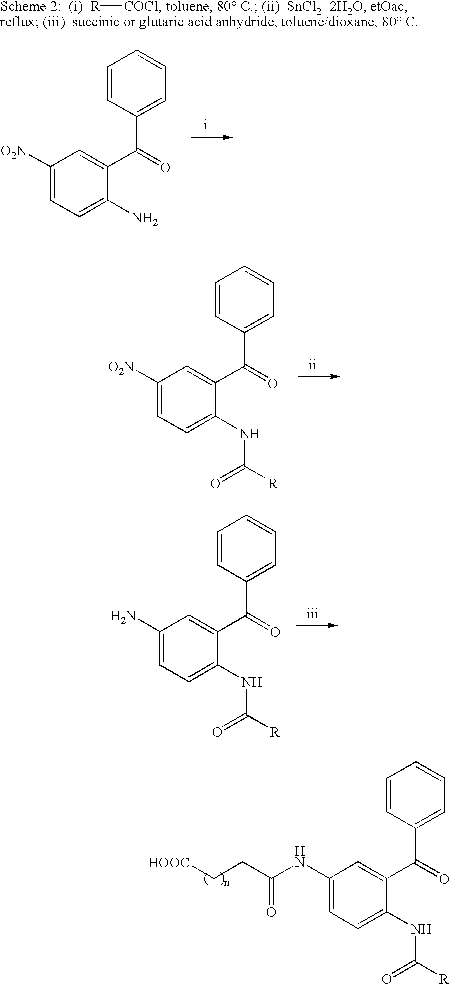

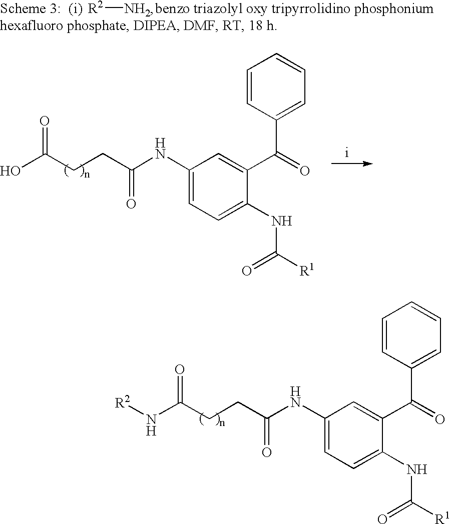

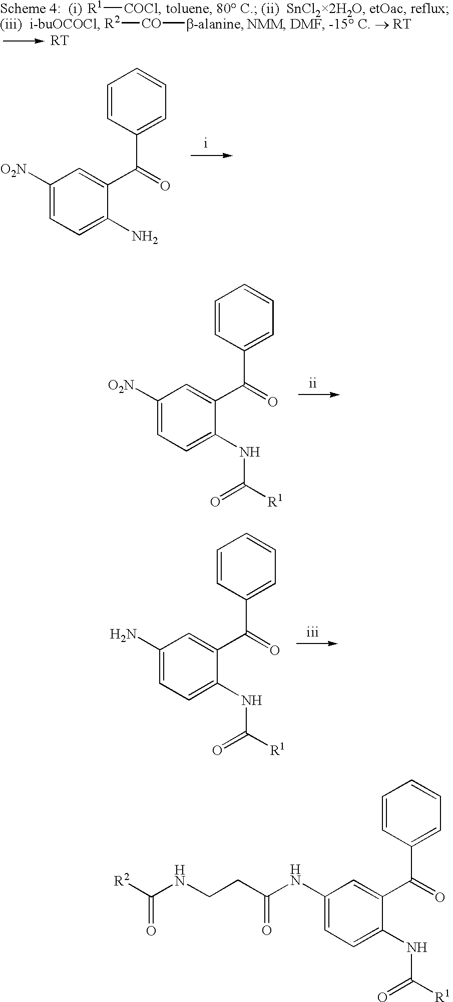

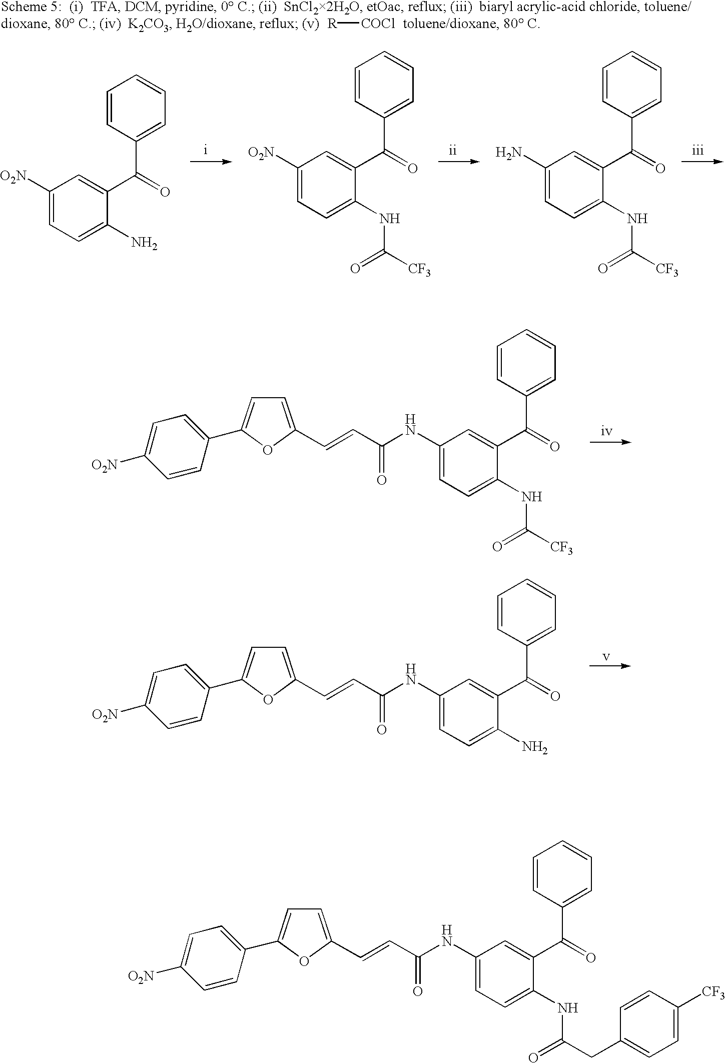

General Instruction 1:

A suitable 2-acyl-4-nitroanilin is dissolved in a sufficient amount toluene—eventually by heating. Subsequently an equimolar amount of a suitable carboxylic acid chloride is added and the mixture is heated to 80° C. for 2 h. Subsequently, the reaction mixture is reduced, whereupon in some cases spontaneous crystallization occurs. The crystals were isolated and are dried in vacuum. If there is no spontaneous crystallization, the solvent is totally removed by distillation and the residue is purified by column chromatography on silica gel.

General Instruction 2:

A solution of a compound obtained according to instruction 1 in ethanol or ethyl acetate (5 ml/mmol) is heated together with tin bichloride dihydrate (5 equivalents 1.125 g/mmol) for 2 h until boiling. The cooled reaction solution is diluted with water, adjusted to pH 7-8 with saturated sodium hydrogen carbonate solution and extracted with ethyl acetate (3×100-200 ml). The combined organic extracts are washed with saturated sodium chloride solution, dried over sodium sulfate and the solvent is totally removed in a rotary evaporator. In general a solid or an oil remains, which often crystallizes within some days.

General Instruction 3:

A solution of one equivalent of a acid chloride in dioxane is added to a solution of a compound obtained according to instruction 2 in toluene/dioxane and the mixture is heated to 80° C. for 1-2 h. Subsequently, it is reduced in vacuum and the obtained solid is isolated.

General Instruction 4:

A solution of one equivalent of an acid anhydride in dioxane is added to a solution of a compound obtained according to instruction 2 compound in toluene/dioxane and the mixture is heated to 80° C. for 1-2 h. Subsequently, it is reduced in the vacuum and the obtained solid is isolated.

General Instruction 5:

One equivalent of 1-benzo triazolyle oxy tripyrrolidino phosphonium hexafluoro phosphate and 3 equivalents of diisopropyl ethylamine are added to a solution of equimolar amounts of an amine and a carboxylic acid in DMF and are stirred at room temperature for 18 h. Subsequently, it is diluted with sodium chloride solution and extracted with ethyl acetate. The extracts are washed with 2N citric acid, saturated solution of sodium hydrogen carbonate and sodium chloride solution. The product remaining after the solvents has been distilled off is purified as stated above.

General Instruction 6:

A suitable N-acylamino acid is dissolved under argon in a sufficient amount of dried DMF and, after addition of 2.28 equivalents of N-methyl—(NMM: 0.25 ml/mmol amino acid) it is cooled to −15° C. Subsequently one equivalent of chloroformic acid isobutyl ester (0.13 ml/mmol amino acid) is added. After five minutes one equivalent of a solution of a compound obtained according to instruction 2 dissolved in a sufficient amount of dried DMF is added to this mixture. The reaction solution is stirred for several hours whereby it slowly reaches room temperature. Subsequently, the composition is poured into a stirred saturated sodium chloride solution (400-800 ml). The watery solution is extracted with ethyl acetate for three times.

The combined extracts are washed with 2 N citric acid, saturated sodium hydrogen carbonate solution and saturated sodium chloride solution and dried over Magnesium sulfate. The residue remaining after removal of the solvents in the rotary evaporator is purified by column chromatography on silica gel.

### EXAMPLE 1

### 2-[N-[3-[3-benzoyl-4-(2-phenylpropionyl)amino]phenylamino]carbamoyl]acetic Acid Methylester

Step 1: N-(2-benzoyl-4-nitrophenyl)-2-phenylpropionic Acid Amide

According to general instruction 1 from 2-amino-5-nitrobenzophenon (1.2 g, 5 mmol) and 2-phenylpropionic acid chloride (0.842 g, 5 mmol). Purification: Recrystallization from ethanol.

yield: 1.098 g (59%)

¹H-NMR (CDCl₃): δ=1.55 (d, J=7 Hz, 3H), 3.74 (q, J=7 Hz, 1H), 7.19 (m, 1H), 7.30 (m, 2H), 7.34 (m, 2H), 7.45 (m, 2H), 7.58 (m, 3H), 8.30 (m, 1H), 8.35 (m, 1H), 8.85 (m, 1H), 11.11 (s, 1H).

Step 2: N-(4-amino-2-benzoylphenyl)-2-phenylpropionic Acid Amide

According to general instruction 2 from N-(2-benzoyl-4-nitrophenyl)-2-phenylpropionic Acid Amide (1.020 g, 2.75 mmol).

yield: 0.937 g (99%)

¹H-NMR (CDCl₃): δ=1.50 (d, J=7 Hz, 3H), 3.63 (q, J=7 Hz, 1H), 6.69 (m, 1H), 6.69 (m 1H), 7.17 (m, 1H), 7.25 (m, 2H), 7.31 (m, 2H), 7.38 (m, 2H), 7.51 (m, 1H), 7.59 (m, 2H), 8.25 (m, 1H), 10.21 (s, 1H).

Step 3: 2-[N-[3-[3-benzoyl-4-(2-phenylpropionyl)amino]phenylamino]carbamoyl]acetic Acid Methylester

C₂₆H₂₄N₂O₅ (444.49 gmol⁻¹)

According to general instruction 3 from N-(4-amino-2-benzoylphenyl)-2-(phenyl) propionic acid amide (0.688 g, 2 mmol) and malonic acid methylester chloride (0.24 ml, 2.2 mmol). column chromatography with ethyl acetate hexane 2:3.

yield: 0.66 g (74%), yellow solid.

IR (KBr): ν=3305, 2920, 1745, 1665, 1560 cm⁻¹. ¹H-NMR (CDCl₃): δ=1.61 (m, 3H), 3.41 (s, 2H), 3.74 (m, 1H), 3.77 (s, 3H), 7.25 (m, 1H), 7.34 (m, 2H), 7.39 (m, 2H), 7.47 (m, 2H), 7.56 (m, 2H), 7.67 (m, 2H), 7.90 (m, 1H), 8.69 (m, 1H), 9.18 (s, 1H), 10.69 (s, 1H)

### EXAMPLE 2

### 2-[N-[3-[3-benzoyl-4-[[2-(4-methylphenyl)acetyl]amino]phenylamino]-carbamoyl]acetic Acid Methylester

Step 1: N-(2-benzoyl-4-nitrophenyl)-2-(4-methylphenyl)acetamide

According to general instruction 1 from 2-amino-5-nitrobenzophenon (1.2 g, 5 mmol) and 2-(4-methylphenyl) acetylchloride (0.843 g, 5 mmol). Purification: Recrystallization from ethanol.

yield 1.75 g (93%).

¹H-NMR (CDCl₃): δ=2.33 (s, 3H), 3.74 (s, 2H), 7.17 (m, 2H), 7.24 (m, 2H), 7.51 (m, 2H), 7.65 (m, 3H), 8.37 (m, 1H), 8.41 (m, 1H), 8.88 (d, J=9 Hz, 1H), 11.05 (s, 1H).

Step 2: N-(4-amino-2-benzoylphenyl)-2-(4-methylphenyl)acetamide

According to general instruction 2 from N-(2-benzoyl-4-nitrophenyl)-2-(4-methylphenyl)acetamide (1.75 g, 4.7 mmol).

yield: 1.053 g (65%).

Step 3: 2-[N-[3-[3-benzoyl-4-[[2-(4-methylphenyl)acetyl]amino]phenylamino]carbamoyl]-acetic Acid Methylester

C₂₆H₂₄N₂O₅ (444.49 gmol¹)

According to general instruction 3 N-(4-amino-2-benzoylphenyl)-2-(4-methylphenyl)acetamide (0.688 g, 2 mmol) and malonic acid methylester chloride (0.24 ml, 2.2 mmol). Recrystallization from toluene.

yield: 0.46 g (50%), yellow solid.

IR (KBr): ν=3295, 2955, 1740, 1690, 1660, 1560 cm⁻¹. ¹H-NMR (CDCl₃): δ=2.22 (s, 3H), 3.32 (s, 2H), 3.40 (s, 2H), 3.62 (s, 3H), 6.95 (m, 2H), 7.01 (m, 2H), 7.45 (m, 2H), 7.54 (m, 1H), 7.62 (m, 4H), 7.73 (m, 1H), 10.0 (s, 1H), 10.22 (s, 1H). ¹³C-NMR (CDCl₃): δ=16.7, 38.4, 39.5, 48.0, 116.5, 118.4, 120.4, 124.3, 124.9, 125.0, 125.6, 127.1, 128.1, 128.3, 128.7, 131.1131.6, 133.3, 160.1, 164.0, 165.2, 191.1.

### EXAMPLE 3

### 3-[N-[3-[3-benzoyl-4-[[2-(4-methylphenyl)acetyl]amino]phenylamino]-carbamoyl]propionic Acid

Steps 1 and 2: s. Example 2

Step 3: 3-[N-[3-[3-benzoyl-4-[[2-(4-methylphenyl)acetyl]aminophenylamino]carbamoyl]-propionic Acid

C₂₆H₂₄N₂O₅ (444.49 gmol⁻¹)

According to general instruction 4 from N-(4-amino-2-benzoylphenyl)-2-(4-methylphenyl)acetamide (0.688 g, 2 mmol) and succinic acid anhydride (0.200 g, 2 mmol). Recrystallization from toluene.

yield: 0.880 g (99%), light yellow solid.

IR (KBr): ν=3330, 2900-2600, 1725, 1655, 1560 cm⁻¹. ¹H-NMR (DMSO-d₆): δ=2.19 (s, 3H), 2.43 (m, 4H), 3.51 (s, 2H), 6.93 (m, 2H), 6.98 (m, 2H), 7.42 (m, 2H), 7.50 (m, 1H), 7.59 (m, 4H), 7.70 (m, 1H), 9.90 (s, 1H), 9.93 (s, 1H). ¹³C-NMR (DMSO-d₆): δ=20.4, 28.6, 30.9, 42.1, 120.0, 121.9, 124.0, 128.0, 128.6, 128.7, 129.3 131.3, 132.1, 132.4, 135.2, 135.3, 137.0, 168.8, 170.0, 173.4, 194.9.

### EXAMPLE 4

### 3-[N-[3-[3-benzoyl-4-[[2-(4-methylphenyl)acetyl]amino]phenylamino]-carbamoyl]propionic Acid Methylester

Steps 1 and 2: s. Example 2

Step 3: 3-[N-[3-[3-benzoyl-4-[[2-(4-methylphenyl)acetyl]amino]phenylamino]carbamoyl]-propionic Acid

C₂₇H₂₆N₂O₅ (458.52 gmol⁻¹)

According to general instruction 3 from N-(4-amino-2-benzoylphenyl)-2-(4-methylphenyl)acetamide (0.344 g, 1 mmol) and succinic acid methylester chloride (0.20 ml, 1 mmol). Re-crystallization from toluene.

yield: 0.348 g (76%), light yellow solid.

IR (KBr): ν=3375, 2950, 2930, 1735, 1715, 1690, 1635, 1550 cm⁻¹. ¹H-NMR (CDCl₃): δ=2.33 (s, 3H), 2.57 (m, 2H), 2.69 (m, 2H), 3.65 (s, 3H), 3.66 (s, 2H), 7.15 (m, 2H), 7.22 (m, 2H), 7.48 (m, 3H), 7.57 (m, 1H), 7.67 (m, 2H), 7.75 (m, 1H), 7.80 (m, 1H), 8.46 (m, 1H), 10.44 (s, 1H). ¹³C-NMR (CDCl₃): δ=21.1, 29.1, 31.8, 45.0, 52.0, 122.4, 124.2, 124.4, 125.1, 128.3, 129.3, 129.6, 130.0 131.2, 132.4, 132.7, 135.2, 136.1, 137.0, 138.0, 169.7, 170.3, 173.6, 198.5.

### EXAMPLE 5

### 4-[N-[3-[3-benzoyl-4-[[2-(4-methylphenyl)acetyl]amino]phenylamino]-carbamoyl]butyric Acid

Steps 1 and 2: s. Example 2

Step 3: 4-[N-[3-[3-benzoyl-4-[[2-(4-methylphenyl)acetyl]amino]phenylamino]carbamoyl]-butyric Acid

C₂₇H₂₆N₂O₅ (458.52 gmol⁻¹)

According to general instruction 6 from N-(4-amino-2-benzoylphenyl)-2-(4-methylphenyl)acetamide (0.688 g, 2 mmol) and glutaric acid anhydride (0.228 g, 2 mmol). Recrystallization from toluene.

yield: 0.740 g (81%), light yellow solid.

IR (KBr): ν=3285, 2900-2600, 1735, 1690, 1660 cm⁻¹. ¹H-NMR (DMSO-d₆): δ=1.76 (m, 2H), 2.22 (m, 5H), 2.28 (m, 2H), 3.31 (s, 2H), 6.95 (m, 2H), 7.01 (m, 2H), 7.45 (m, 2H), 7.51-(m, 1H), 7.62 (m, 4H), 7.75 (m, 1H), 9.91 (s, 1H), 9.93 (s, 1H), 11.91 (s, 1H). ¹³C-NMR (DMSO-d₆): δ=20.2, 20.4, 32.8, 35.2, 42.5, 120.1, 122.0, 124.0, 128.0, 128.6, 128.7, 129.3 131.3, 132.1, 132.4, 135.2, 135.3, 137.0, 168.8, 170.6, 173.7, 194.3.

### EXAMPLE 6

### 4-[N-[3-[3-benzoyl-4-[[2-(4-methylphenyl)acetyl]amino]phenylamino]-carbamoyl]butyric Acid Methylester

Steps 1 and 2: s. Example 2

Step 3: 4-[N-[3-[3-benzoyl-4-[[2-(4-methylphenyl)acetyl]amino]phenylamino]carbamoyl]-butyric Acid Methylester

C₂₈H₂₈N₂O₅ (472.55 gmol⁻¹)

According to general instruction 3 from N-(4-amino-2-benzoylphenyl)-2-(4-methylphenyl)acetamide (0.344 g, 1 mmol) and glutaric acid methylesterchloride (0.17 ml, 1 mmol). Re-crystallization from toluene.

yield: 0.310 g (65%), light yellow solid.

IR (KBr): ν=3300, 3050, 2950, 1740, 1660, 1560 cm⁻¹. ¹H-NMR (CDCl₃): δ=1.90 (m, 2H), 2.25 (s, 3H), 2.27 (m, 4H), 3.56 (s, 3H), 3.59 (s, 2H), 7.08 (m, 2H), 7.15 (m, 2H), 7.40 (m, 3H), 7.50 (m, 1H), 7.61 (m, 3H), 7.78 (m, 1H), 8.38 (m, 1H), 10.38 (s, 1H). ¹³C-NMR (CDCl₃): δ=20.5, 21.1, 32.9 36.1, 45.0, 51.6, 122.4, 124.3, 124.4, 125.1, 128.3, 129.3, 129.6, 130.0 131.1, 132.6, 132.7, 136.0, 137.0, 138.0, 170.3, 170.5, 173.7, 198.5.

### EXAMPLE 7

### N-[2-[3-benzoyl-4-[[2-(4-methylphenyl)acetyl]amino]phenylamino]-2-oxoethyl]-heptadecanoic Acid Amide

Steps 1 and 2: s. Example 2

Step 3: N-[2-[3-benzoyl-4-[[2-(4-methylphenyl)acetyl]amino]phenylamino]-2-oxoethyl]-heptadecanoic acid amide C₄₁H₅₅N₃O₄ (653.91 gmol⁻¹)

According to general instruction 6 from N-(4-amino-2-benzoylphenyl)-2-(4-methylphenyl)acetamide (0.344 g, 1 mmol) and N-heptadecanoylglycine (0.328 g, 1 mmol). column chromatography with ethyl acetate n-hexane 3:2.

yield: 0.415 g (63%), yellow solid, solid.t.: 46° C.

Cal.: C 75.31, H 8.48, N 6.43; Found: C, 75.34H, 8.79 N 6.56.

IR (KBr): ν=3305, 2920, 2850, 1645, 1555 cm⁻¹. ¹H-NMR (CDCl₃): δ=0.80 (t, J=7 Hz, 3H), 1.21 (m, 26H), 1.47 (m, 2H), 2.08 (m, 2H), 2.26 (s, 3H), 3.61 (s, 2H), 3.99 (d, J=5 Hz, 2H), 6.44 (m, 1H), 7.10 (m, 2H), 7.18 (m, 3H), 7.39 (m, 2H), 7.50 (m, 2H), 7.61 (m, 2H), 7.78 (m, 1H), 8.43 (m, 1H), 8.95 (s, 1H), 10.42 (s, 1H). ¹³C-NMR (CDCl₃): δ=14.1, 21.1, 22.7, 24.8, 25.5, 29.1, 29.2, 29.3, 29.4, 29.5, 29.6, 29.7, 29.8, 31.9, 33.7, 36.3, 44.4, 45.0, 122.4, 124.1, 124.2, 125.1, 128.3, 129.3, 129.6, 130.0, 131.1, 132.3, 132.7, 136.3, 137.0, 138.0, 167.1 170.3, 174.4, 198.4. MS: m/z=652 (100, M⁺), 344 (93), 212 (94).

### EXAMPLE 8

### N-[4-[3-benzoyl-4-[[2-(4-methylphenyl)acetyl]amino]phenylamino]-4-oxobutyl]-pentadecanoic Acid Amide

Steps 1 and 2: s. Example 2

Step 3: N-[4-[3-benzoyl-4-[[2-(4-methylphenyl)acetyl]amino]phenylamino]-4-oxobutyl]-pentadecanoic acid amide

C₄₁H₅SN₃O₄ (653.91 gmol⁻¹)

According to general instruction 6 from N-(4-amino-2-benzoylphenyl)-2-(4-methylphenyl)acetamide (0.688 g, 2 mmol) and N-pentadecanoyl-γ-amino butyric acid (0.712 g, 2 mmol). column chromatography with ethyl acetate n-hexane 3:2.

yield: 0.310 g (47%), yellow solid, solid.t.: 115° C.

Cal.: C 75.31, H 8.48, N 6.43; Found: C, 74.91H, 8.06 N 6.62.

IR (KBr): ν=3295, 2920, 2850, 1645, 1550 cm⁻¹. ¹H-NMR (CDCl₃): δ=0.81 (t, J=7 Hz, 3H), 1.20 (m, 22H), 1.50 (m, 2H), 1.75 (m, 2H), 2.07 (m, 2H), 2.23 (m, 2H), 2.26 (s, 3H), 3.24 (m, 2H), 3.60 (s, 2H), 5.80 (m, 1H), 7.10 (m, 2H), 7.17 (m, 3H), 7.39 (m, 2H), 7.50 (m, 2H), 7.65 (m, 2H), 7.96 (m, 1H), 8.43 (m, 1H), 9.20 (s, 1H), 10.46 (s, 1H). ¹³C-NMR (CDCl₃): δ=14.1, 21.1, 22.7, 25.8, 26.6, 29.2, 29.3, 29.4, 29.5, 29.6, 29.63, 29.7, 31.9, 34.6, 36.8, 38.5, 45.1, 122.2, 124.1, 124.4, 125.2, 128.3, 129.3, 129.6, 130.1, 131.3, 132.6, 133.1, 136.0, 136.9, 138.1, 170.2, 171.2, 174.7, 198.8. MS: m/z=652 (18, M⁺), 140 (64), 127 (100).

### EXAMPLE 9

### 4-[N-[3-[3-benzoyl-4-[[2-(4-methylphenyl)acetyl]amino]phenylamino]-carbamoyl]butyric Acid-N-tetradecylamide

C₄₁H₅₅N₃O₄ (653.91 gmol⁻¹)

According to general instruction 5 from tetradecylamine (0.220 g, 1.0 mmol) 3-[N-[3-[3-benzoyl-4-[[2-(4-methylphenyl)acetyl]amino]phenylamino]carbamoyl]butyric acid (0.460 g, 1.0 mmol). Purification: column chromatography on silica gel with etac:n-hexane 3:2.

yield: 0.325 g (50%), yellow solid, solid.t.: 109° C.

Cal.: C 75.31, H 8.48, N 6.43; Found: C, 75.04H, 8.23 N 6.72.

IR (KBr): ν=3300, 3060, 2925, 2855, 1655, 1550 cm⁻¹. ¹H-NMR (CDCl₃): δ=0.81 (t, J=7 Hz, 3H), 1.18 (m, 20H), 1.39 (m, 2H), 1.90 (m, 2H), 2.18 (m, 2H), 2.27 (s, 3H), 2.32 (m, 2H), 3.13 (m, 2H), 3.60 (s, 2H), 5.55 (m, 1H), 7.10 (m, 2H), 7.18 (m, 4H), 7.41 (m, 2H), 7.51 (m, 2H), 7.64 (m, 2H), 7.83 (m, 1H), 8.43 (m, 1H), 10.42 (s, 1H). ¹³C-NMR (CDCl₃): δ=14.1, 21.1, 22.7, 26.9, 29.2, 29.3, 29.5, 29.56, 29.6, 31.9, 35.0, 36.3, 39.7, 45.0, 122.3, 124.3, 125.1, 128.3, 129.3, 129.6, 130.0, 132.7, 132.8, 138.1, 170.2, 171.1, 172.7, 198.7.

### EXAMPLE 10

### N-[3-benzoyl-4-[[2-(4-methylphenyl)acetyl]amino]phenyl]eicosanoic Acid Amide

Steps 1 and 2: s. Example 2

Step 3: N-[3-benzoyl-4-[[2-(4-methylphenyl)acetyl]amino]phenyl]eicosanoic Acid Amide

C₄₂H₅₉N₂O₃ (638.94 gmol⁻¹)

According to general instruction 6 from N-(4-amino-2-benzoylphenyl)-2-(4-methylphenyl)acetamide (0.344 g, 1 mmol) and eicosanoic acid (0.312 g, 1 mmol). column chromatography with ethyl acetate n-hexane 2:3.

yield: 0.476 g (75%), yellow solid, solid.t.: 102° C.

Cal.: C 78.95, H 9.15, N 4.38; Found: C, 78.62H, 8.78 N 4.63.

IR (KBr): ν=3290, 2920, 2850, 1685, 1655, 1550 cm⁻¹. ¹H-NMR (CDCl₃): δ=0.81 (t, J=7 Hz, 3H), 1.20 (m, 32H), 1.58 (m, 2H), 2.20 (m, 2H), 2.27 (s, 3H), 3.61 (s, 2H), 7.09 (m, 2H), 7.18 (m, 3H), 7.41 (m, 2H), 7.50 (m, 1H), 7.64 (m, 2H), 7.79 (m, 1H), 8.43 (m, 1H), 10.41 (s, 1H). ¹³C-NMR (CDCl₃): δ=14.1, 19.0, 21.1, 22.7, 25.8, 29.2, 29.3, 29.4, 29.44, 29.5, 29.6, 29.7, 31.9, 37.6, 45.0, 122.4, 124.3, 124.4, 125.1, 128.3, 129.3, 129.6, 130.0, 132.4, 132.7, 136.2, 137.0, 138.0, 170.3, 171.4, 198.6. MS: m/z=638 (100, M⁺), 506 (68).

### EXAMPLE 11

### N-[3-[3-benzoyl-4-[(2-phenylacetyl)amino]phenylamino]-3-oxopropyl]-hexadecanoic Acid Amide

Step 1: N-(2-benzoyl-4-nitrophenyl)-2-phenyl Acetamide

According to general instruction 1 from 2-amino-5-nitrobenzophenon (1.2 g, 5 mmol) and phenyl acetyl chloride (0.8 ml, 5 mmol).

yield 1.7 g (94%).

¹H-NMR (CDCl₃): δ=3.79 (s, 2H), 7.37 (m, 2H), 7.52 (m, 3H), 7.64 (m, 3H), 8.15 (m, 1H), 8.25 (m, 1H), 8.41 (m, 1H), 8.46 (m, 1H), 8.98 (m, 1H), 11.08 (s, 1H).

Step 2: N-(4-amino-2-benzoylphenyl)-2-phenyl Acetamide

According to general instruction 2 from N-(2-benzoyl-4-nitrophenyl)-2-phenyl acetamide (1.7 g, 4.7 mmol).

yield 1.38 g (89%).

¹H-NMR (CDCl₃): δ=3.53 (s, 2H), 3.61 (s, 2H), 6.68 (m, 1H), 6.79 (m, 1H), 7.18-7.30 (m, 5H), 7.36-7.40 (m, 2H), 7.51 (m, 1H), 7.62 (m, 2H), 8.21 (m, 1H), 10.05 (s, 1H).

Step 3: N-[3-[3-benzoyl-4-[(2-phenylacetyl)amino]phenylamino]-3-oxopropyl]hexadecanoic Acid Amide

C₄₀H₅₃N₃O₄ (639.89 gmol⁻¹)

According to general instruction 6 from N-hexadodecanoyl-β-alanine (0.490 g, 1.5 mmol) and N-(4-amino-2-benzoylphenyl)-2-phenyl acetamide (0.495 g, 1.5 mmol). Purification: Column chromatography on silica gel with 1. ethyl acetate-n-hexane 2:3 and 2. ethyl acetate as eluant.

yield: 0.85 g (88%), yellow solid, solid.t.: 117° C.

Cal.: C 75.08, H 8.35, N 6.57; Found: C, 74.77H, 8.38 N 6.89.

IR (KBr): ν=3310, 2920, 2850, 1640, 1550 cm⁻¹. ¹H-NMR (CDCl₃): δ=0.81 (t, J=7 Hz, 3H), 1.17 (m, 24H), 1.45 (m, 2H), 2.02 (t, J-7 Hz, 2H), 2.46 (m, 2H), 3.44 (m, 2H), 3.65 (s, 2H), 6.12 (m, 1H), 7.23 (m, 1H), 7.29 (m, 3H), 7.39 (m, 2H), 7.48-7.57 (m, 2H), 7.62 (m, 2H), 7.77 (m, 1H), 8.43 (m, 2H), 10.45 (s, 1H). ¹³C-NMR (CDCl₃): δ=14.1, 22.7, 25.6, 29.2, 29.3, 29.4, 29.5, 29.6, 29.62, 29.7, 31.9, 35.2, 36.7, 36.9, 45.4, 122.4, 124.2, 124.3, 125.3, 127.4, 128.3, 128.9, 129.4, 130.0, 132.6, 132.7, 134.2, 136.2, 138.0, 169.7, 170.0, 174.0, 198.6. MS: m/z=639 (100, M⁺), 330 (69), 312 (67), 212 (73).

### EXAMPLE 12

### N-[2-[3-benzoyl-4-[[2-(4-methylphenyl)acetyl]amino]phenylamino]-2-oxo-ethyl]-4-phenylcinnamic Acid Amide

Steps 1 and 2: s. Example 2

Step 3: N-[2-[3-benzoyl-4-[[2-(4-methylphenyl)acetyl]amino]phenylamino]-2-oxoethyl]-4-phenylcinnamic Acid Amide

C₄₀H₃₅N₃O₄ (607.72 gmol⁻¹)

According to general instruction 6 from N-(4-phenylcinnamoyl) glycine (0.282 g, 1.0 mmol) and N-(4-amino-2-benzoylphenyl)-2-(4-methylphenyl) acetamide (0.344 g, 1.0 mmol). Purification: column chromatography on silica gel with 1. ethyl acetate n-hexane 3:2, 2. ethyl acetate.

yield: 0.476 g (78%), yellow solid, solid.t.: 223° C.

Cal.: C 77.08, H, 5.47 N, 6.92; Found: C 76.75, H 5.95, N, 6.66.

IR (KBr): ν=3275, 3030, 2925, 1655, 1610, 1510, cm⁻¹. ¹H-NMR (DMSO-d₆): δ=2.22 (s, 3H), 3.33 (s, 2H), 3.98 (m, 2H), 6.77 (d, J=16 Hz, 1H), 6.98 (m, 2H), 7.02 (m, 2H), 7.18 (d, J=16 Hz, 1H), 7.35 (m, 2H), 7.45 (m, 6H), 7.68 (m, 10H), 7.75 (m, 2H), 8.32 (m, 1H), 10.0 (s, 1H), 10.10 (s, 1H). ¹³C-NMR (DMSO-d₆): δ=20.9, 42.5, 43.0, 119.1, 120.8, 122.2, 122.7, 124.5, 126.8, 126.9, 127.2, 127.4, 128.0, 128.4, 128.5, 128.8, 129.1, 129.2, 129.3, 129.8, 132.1, 132.5, 132.8, 134.3, 134.7, 135.3, 135.7, 137.5, 138.8, 139.7, 139.8, 140.5, 141.4, 165.7, 168.1, 169.3, 195.4.

### EXAMPLE 13

### N-[3-[3-benzoyl-4-[(2-phenylacetyl)amino]phenylamino]-3-oxopropyl]-4-benzyl-oxycinnamic Acid Amide

Steps 1 and 2: s. Example 11

Step 3: N-[3-[3-benzoyl-4-[(2-phenylacetyl)amino]phenylamino]-3-oxopropyl]-4-benzyl-oxycinnamic Acid Amide

C₄₀H₃₅N₃O₅ (637.74 gmol⁻¹)

According to general instruction 6 from N-(4-benzyloxycinnamoyl)-p-alanine (0.44 g, 1.35 mmol) and N-(4-amino-2-benzoylphenyl)-2-phenyl acetamide (0.445 g, 1.35 mmol). Purification: column chromatography on silica gel with 1. ethyl acetate n-hexane 3:2 2. ethyl acetate as eluant.

yield: 0.8 g (93%), yellow solid, solid.t.: 138° C.

Cal.: C 75.34, H, 5.53 N, 6.59; Found: C 74.95, H 5.72, N, 6.22.

IR (KBr): ν=3300, 3090, 2925, 1710, 1685, 1655, 1615, 1610, 1510, cm⁻¹. ¹H-NMR (CDCl₃): δ=2.54 (m, 2H), 3.55 (m, 2H), 3.64 (s, 2H), 4.99 (s, 2H), 6.13 (d, J=16 Hz, 1H), 6.43 (m, 1H), 6.85 (m, 2H), 7.21-7.46 (m, 17H), 7.58 (m, 2H), 7.76 (m, 1H), 8.43 (m, 1H), 8.58 (s, 1H), 10.45 (s, 1H). ¹³C-NMR (CDCl₃): δ=37.7, 37.1,45.5, 70.1, 109.4, 115.2117.8, 122.5, 124.3, 124.6, 125.5, 127.4, 127.5, 128.2, 128.4, 128.7, 129.0, 129.5, 130.1, 132.7, 132.8, 134.3, 136.3, 136.6, 138.1, 141.2, 160.3, 167.2, 170.0, 170.1, 198.7. MS: m/z=637 (0.2, M⁺), 384 (14), 312 (21), 311 (22), 253 (27), 91 (100).

### EXAMPLE 14

### N-[3-benzoyl-4-[2-(4-methylphenyl)acetyl]amino]phenylnicotinic Acid Amide

Steps 1 and 2: s. Example 2

Step 3: N-[3-benzoyl-4-[2-(4-methylphenyl)acetyl]amino]phenyl]nicotinic acid amide C₂₈H₂₃N₃O₃ (449.51 gmol⁻¹)

According to general instruction 3 from nicotinic acid chloride (0.142 g, 1.0 mmol) and N-(4-amino-2-benzoylphenyl)-2-(4-methylphenyl) acetamide (0.344 g, 1.0 mmol). Purification: column chromatography on silica gel with ethyl acetate n-hexane 3:2.

yield: 0.098 g (22%), yellow solid, solid.t.: 198° C.

Cal.: C 74.82, H, 5.16 N, 9.35; Found: C 74.66, H 5.39, N, 9.05.

IR (KBr): ν=3400, 2925, 1675, 1635, 1595, 1555 cm⁻¹. ¹H-NMR (CDCl₃): δ=2.26 (s, 3H), 3.60 (s, 2H), 7.08 (m, 2H), 7.15 (m, 2H), 7.30 (m, 1H), 7.41 (m, 2H), 7.51 (m, 1H), 7.58 (m, 1H), 7.64 (m, 2H), 7.91 (m, 1H), 8.14 (m, 1H), 8.47 (m, 1H), 8.65 (s, 1H), 8.97 (s, 1H), 10.44 (s, 1H). ¹³C-NMR (CDCl₃): δ=21.1, 45.1, 12.6, 123.8, 124.4, 125.1, 125.9, 128.4, 129.3, 129.7, 130.0, 130.3, 131.0, 132.0, 132.8, 135.2, 136.8, 137.1, 138.0, 147.9, 152.6, 163.9, 170.5, 198.4. MS: m/z=449 (75, M⁺), 317 (100).

### EXAMPLE 15

### N-[3-benzoyl-4-[2-(4-methylphenyl)acetyl]amino]phenyl]benzoic Acid Amide

Steps 1 and 2: s. Example 2

Step 3: N-[3-benzoyl-4-[2-(4-methylphenyl)acetyl]amino]phenyl]benzoic Acid Amide

C₂₉H₂₄N₂O₃ (448.53 gmol⁻¹)

According to general instruction 3 from benzoic acid chloride (0.14 g, 1.0 mmol) and N-(4-amino-2-benzoylphenyl)-2-(4-methylphenyl)acetamide (0.344 g, 1.0 mmol). Purification: Recrystallization from toluene.

yield: 0.365 g (81%), yellow solid, solid.t.: 212° C.

Cal.: C 77.66, H, 5.36 N, 6.25; Found: C 77.40, H 5.29, N, 6.38.

IR (KBr): ν=3420, 1650, 1620, 1550, 1500 cm⁻¹. ¹H-NMR (CDCl₃): δ=2.34 (s, 3H), 3.66 (s, 2H), 7.14 (m, 2H), 7.22 (m, 2H), 7.40-7.50 (m, 5H), 7.57 (m, 1H), 7.62 (m, 1H), 7.70 (m, 2H), 7.78 (m, 2H), 7.90 (m, 1H), 7.98 (m, 1H), 8.55 (m, 1H), 10.52 (s, 1H). MS: m/z=448 (96, M⁺), 316 (100).

### EXAMPLE 16

### N-[3-benzoyl-4-[2-(4-methylphenyl)acetyl]amino]phenyl]-2-phenylacetic Acid-Acid Amide

Steps 1 and 2: s. Example 2

Step 3: N-[3-benzoyl-4-[2-(4-methylphenyl)acetyl]amino]phenyl]-2-phenylacetic Acid Amide

C₃₀H₂₆N₂O₃ (462.58 gmol⁻¹)

According to general instruction 3 from phenylacetic acid chloride (0.2 ml, 1.5 mmol) and N-(4-amino-2-benzoylphenyl)-2-(4-methylphenyl)acetamide (0.516 g, 1.5 mmol). Purification: Recrystallization from toluene.

yield: 0.471 g (68%), yellow solid, solid.t.: 134° C.

Cal.: C 77.90, H, 5.67 N, 6.06; Found: C 77.51, H 5.50, N, 6.48.

IR (KBr): ν=3290, 1700, 1670, 1630 cm⁻¹. ¹H-NMR (CDCl₃): δ=2.30 (s, 3H), 3.61 (s, 2H), 3.64 (s, 2H), 7.13 (m, 2H), 7.20 (m, 2H), 7.25-7.45 (m, 6H), 7.57 (m, 1H), 7.43 (m, 2H), 7.55 (m, 1H), 7.65 (m, 2H), 7.87 (m, 1H), 8.43 (m, 1H), 10.44 (s, 1H). MS: m/z=462 (100, M⁺), 330 (79).

### EXAMPLE 17

### N-[3-benzoyl-4-[2-(4-methylphenyl)acetyl]amino]phenyl]-3-phenylpropion-acid Amide

Steps 1 and 2: s. Example 2

Step 3: N-[3-benzoyl-4-[2-(4-methylphenyl)acetyl]amino]phenyl]-3-phenylpropionic Acid-Amid

C₃₁H₂₈N₂O₃ (476.58 gmol⁻¹)

According to general instruction 3 from 3-Phenylpropionic acid chloride (0.17 ml, 1.0 mmol) and N-(4-amino-2-benzoylphenyl)-2-(4-methylphenyl)acetamide (0.344 g, 1.0 mmol). Purification: Recrystallization from toluene.

yield: 0.070 g (15%), yellow solid, solid.t.: 59° C.

Cal.: C 78.18, H, 5.92 N, 5.88; Found: C 78.22, H 5.91, N, 5.78.

IR (KBr): ν=3420, 3270, 1655, 1595, 1555, 1505 cm⁻¹. ¹H-NMR (CDCl₃): δ=2.32 (s, 3H), 2.56 (t, J=8 Hz, 2H), 2.96 (t, J=8 Hz, 2H), 3.66 (s, 2H), 7.14 (m, 5H), 7.22 (m, 5H), 7.35 (m, 1H), 7.47 (m, 2H), 7.58 (m, 1H), 7.66 (m, 2H), 7.75 (m, 1H), 8.44 (m, 1H), 10.47 (s, 1H). ¹³C-NMR (CDCl₃): δ=21.1, 31,5, 39.3, 45.0. 122.4, 124.3, 124.6, 125.4, 126.4, 128.3, 128.4, 128.6, 129.3, 129.6, 130.0, 131.1, 132.2, 132.7, 136.0, 137.0, 138.0, 140.4, 170.3, 170.4, 198.5. MS: m/z=476 (57, M⁺), 458 (100).

### EXAMPLE 18

### N-[3-benzoyl-4-[2-(4-methylphenyl)acetyl]amino]phenyl]-3-cyclohexyl Propionic Acid Amide

Steps 1 and 2: s. Example 2

Step 3: N-[3-benzoyl-4-[2-(4-methylphenyl)acetyl]amino]phenyl]-3-cyclohexyl Propionic Acid Amide

C₃₁H₃₄N₂O₃ (482.63 gmol⁻¹)

According to general instruction 3 from 3-cyclohexyl propionic acid chloride (0.17 ml, 1.0 mmol) and N-(4-amino-2-benzoylphenyl)-2-(4-methylphenyl) acetamide (0.344 g, 1.0 mmol). Purification: Recrystallization from toluene.

yield: g (%), yellow solid, solid.t.: 55° C.

Cal.: C 77.15, H, 7.10 N, 5.80; Found: C 77.07, H 6.91, N, 5.55.

IR (KBr): ν=3285,2925, 2850, 1660, 1550, 1510 cm⁻¹. ¹H-NMR (CDCl₃): δ=1.14-1.25 (m, 4H), 1.55 (m, 2H), 1.67 (m, 6H), 2.27 (m, 2H), 2.34 (m, 4H), 3.66 (s, 2H). MS: m/z=482 (100, M⁺), 350 (57).

### EXAMPLE 19

### N-[3-benzoyl-4-[2-(4-methylphenyl)acetyl]amino]phenyl]-4-phenylbutyric Acid Amide

Steps 1 and 2: s. Example 2

Step 3: N-[3-benzoyl-4-[2-(4-methylphenyl)acetyl]amino]phenyl]-4-phenylbutyric Acid Amide

C₃₂H₃₀N₂O₃ (490.61 gmol⁻¹)

According to general instruction 3 from 4-phenylbutyric acid chloride (0.185 mg, 1.0 mmol) and N-(4-amino-2-benzoylphenyl)-2-(4-methylphenyl) acetamide (0.344 g, 1.0 mmol). Purification: column chromatography ethyl acetate n-hexane 2:3.

yield: 0.405 g (83%), yellow solid, solid.t.: 117° C.

Cal.: C 78.34, H, 6.16 N, 5.71; Found: C 78.42, H 5.84, N, 6.02.

IR (KBr): ν=3430, 3290, 2945, 1700, 1645 cm⁻¹. ¹H-NMR (CDCl₃): δ=1.94 (m, 2H), 2.20 (m, 2H), 2.26 (s, 3H), 2.60 (m, 2H), 3.60 (s, 2H), 7.10 (m, 5H), 7.17 (m, 5H), 7.41 (m, 3H), 7.53 (m, 1H), 7.61 (m, 2H), 7.72 (m, 1H), 8.44 (m, 1H), 10.41 (s, 1H). MS: m/z=490 (100, M⁺), 358 (52).

### EXAMPLE 20

### N-[3-benzoyl-4-[2-(4-methylphenyl)acetyl]amino]phenyl]-5-phenylvaleric Acid Amide

Steps 1 and 2: s. Example 2

Step 3: N-[3-benzoyl-4-[2-(4-methylphenyl)acetyl]amino]phenyl]-5-phenylvaleric Acid Amide

C₃₁H₂₈N₂O₃ (504.63 gmol⁻¹)

According to general instruction 3 from 5-phenylvaleric acid chloride (0.200 mg, 1.0 mmol) and N-(4-amino-2-benzoylphenyl)-2-(4-methylphenyl) acetamide (0.344 g, 1.0 mmol). Purification: column chromatography ethyl acetate n-hexane 2:3.

yield: 0.245 g (49%), yellow solid.

¹H-NMR (CDCl₃): δ=1.61 (m, 4H), 2.21 (m, 2H), 2.26 (s, 3H), 2.55 (m, 2H), 3.60 (s, 2H), 7.09 (m, 5H), 7.18 (m, 5H), 7.40 (m, 3H), 7.51 (m, 1H), 7.62 (m, 2H), 7.76 (m, 1H), 8.41 (m, 1H), 10.41 (s, 1H).

### EXAMPLE 21

### N-[3-benzoyl-4-[2-(4-methylphenyl)acetyl]amino]phenyl]cinnamic Acid Amide

Steps 1 and 2: s. Example 2

Step 3: N-[3-[3-benzoyl-4-[2-(4-methylphenyl)acetyl]amino]phenylamino]cinnamic Acid Amide

C₃₁H₂₆N₂O₃ (474.56 gmol⁻¹)

According to general instruction 3 from cinnamic acid chloride (0.210 g, 1.0 mmol) and N-(4-amino-2-benzoylphenyl)-2-(4-methylphenyl) acetamide (0.270 g, 1.0 mmol). Purification: Recrystilization from toluene.

yield: 0.214 g (45%), yellow solid, solid.t.: 87° C.

Cal.: C 78.46, H, 5.52 N, 5.90; Found: C 78.82, H 5.54, N, 5.86.

IR (KBr): ν=3440, 3260, 3085, 1665, 1635, 1505 cm⁻¹. ¹H-NMR (CDCl₃): δ=2.31 (s, 3H), 3.67 (s, 2H), 6.45 (d, J=16 Hz, 1H), 7.15 (m, 3H), 7.22 (m, 3H), 7.34 (m, 3H), 7.46 (m, 4H), 7.56 (m, 2H), 7.68 (m, 2H), 8.01 (m, 1H), 8.51 (m, 1H), 10.51 (s, 1H). MS: m/z=474 (100, M⁺), 342 (39).

### EXAMPLE 22

### N-[3-benzoyl-4-[2-(4-methylphenyl)acetyl]amino]phenyl]-4-nitrocinnamic Acid Amide

Steps 1 and 2: s. Example 2

Step 3: N-[3-[3-benzoyl-4-[2-(4-methylphenyl)acetyl]amino]phenylamino]cinnamic Acid Amide

C₃₁H₂₅N₃O₅ (519.56 gmol⁻¹)

According to general instruction 3 from 4-nitrocinnamic acid chloride (0.382 g, 1.5 mmol) and N-(4-amino-2-benzoylphenyl)-2-(4-methylphenyl) acetamide (0.516 g, 1.5 mmol). Purification: Recrystallization from toluene.

yield: 0.560 g (72%), yellow solid, solid.t.: 224° C.

Cal.: C 71.28, H, 4.59 N, 8.31; Found: C 71.36, H 4.73, N, 8.31.

IR (KBr): ν=3430, 3070, 1685, 1665, 1505 cm⁻¹. ¹H-NMR (DMSO-d₆): δ=2.22 (s, 3H), 3.35 (s, 2H), 6.90 (d, J=18 Hz, 1H), 7.00 (m, 4H), 7.48 (m, 3H), 7.61 (m, 3H), 7.66 (m, 2H), 7.75 (m, 1H), 7.82 (m, 2H), 8.22 (m, 2H), 10.00 (s, 1H), 10.37 (s, 1H). MS: m/z=519 (68, M⁺), 387 (100), 212 (75).

### EXAMPLE 23

### N-[3-[3-benzoyl-4-[2-(4-methylphenyl)acetyl]amino]phenylamino]-4-phenyl-cinnamic Acid Amide

Steps 1 and 2: s. Example 2

Step 3: N-[3-[3-benzoyl-4-[2-(4-methylphenyl)acetyl]amino]phenylamino]-4-phenyl-cinnamic Acid Amide

C₃₇H₃₀N₂O₃ (550.66 gmol⁻¹)

According to general instruction 3 from 4-phenylcinnamic acid chloride (0.282 g, 1.0 mmol) and N-(4-amino-2-benzoylphenyl)-2-(4-methylphenyl) acetamide (0.270 g, 1.0 mmol). Purification: Recrystallization from toluene.

yield: 0.456 g (83%), yellow solid, solid.t.: 200° C.

Cal.: C 80.70, H, 5.49 N, 5.09; Found: C 80.53, H 5.36, N, 5.12.

IR (KBr): ν=3445, 1700, 1685, 1655, 1640, 1550, 1505 cm⁻¹. ¹H-NMR (CDCl₃): δ=2.24 (s, 3H), 3.60 (s, 2H), 6.42 (d, J=16 Hz, 1H), 7.08 (m, 2H), 7.16 (m, 3H), 7.28 (m, 1H), 7.38 (m, 5H), 7.48 (m, 6H), 7.61 (m, 3H), 7.96 (m, 1H), 8.43 (m, 1H), 10.46 (s, 1H). MS: m/z=550 (98, M⁺), 207 (100).

### EXAMPLE 24

### N-[2-[3-benzoyl-4-[[2-(4-methylphenyl)acetyl]amino]phenyl]-4-benzyloxy-cinnamic Acid Amide

Steps 1 and 2: s. Example 2

Step 3: N-[2-[3-benzoyl-4-[[2-(4-methylphenyl)acetyl]amino]phenyl]-4-benzyloxycinnamic Acid Amide

C₃₈H₃₂N₂O₄ (580.72 gmol⁻¹)

According to general instruction 3 from 4-benzyloxycinnamic acid chloride (0.327 g, 1.2 mmol) and N-(4-amino-2-benzoylphenyl)-2-(4-methylphenyl) acetamide (0.413 g, 1.2 mmol). Purification: Recryst. from n-hexane/acetone. is yield: 0.482 g (69%), yellow solid, solid.t.: 183° C.

Cal.: C 78.60, H 5.55, N 4.82. Found: C 78.85, H 5.59, N, 4.94.

IR (KBr): ν=3285, 1675, 1600, 1540 cm⁻¹. ¹H-NMR (CDCl₃): δ=2.31 (s, 3H), 3.67, (s, 2H), 5.06 (s, 2H), 6.33 (d, J=16 Hz, 1H), 6.91 (m, 2H), 7.15 (m, 2H), 7.24 (m, 2H), 7.34 (m, 9H), 7.55 (m, 2H), 7.60 (d, J=16 Hz, 1H), 7.70 (m, 2H), 7.78,(s, 1H), 8.03 (s, 1H), 8.49 (m, 1H), 10.52 (s, 1H). ¹³C-NMR (CDCl₃): δ=21.1, 45.1, 70.1, 115.2, 118.0, 122.4, 124.4, 125.2, 128.3, 128.7, 129.3, 129.6, 129.6, 130.0, 131.1, 132.7, 132.9, 136.2, 136.5, 137.0, 138.1, 142.2, 160.4, 164.4, 170.4, 198.7. MS: m/z=580 (0.3, M⁺), 581 (81), 345 (55), 237 (100).

### EXAMPLE 25

### N-[2-[3-benzoyl-4-[[2-(4-methylphenyl)acetyl]amino]phenylamino]-2-oxo-ethyl]-4-benzoic Acid Amide

Steps 1 and 2: s. Example 2

Step 3: N-[2-[3-benzoyl-4-[[2-(4-methylphenyl)acetyl]amino]phenylamino]-2-oxoethyl]-4-benzoic Acid Amide

C₃₁H₂₇N₃O₄ (505.61 gmol⁻¹)

According to general instruction 6 from N-(benzoyl)glycine (0.18 g, 1.0 mmol) and N-(4-amino-2-benzoylphenyl)-2-(4-methylphenyl)acetamide (0.344 g, 1.0 mmol). Purification: column chromatography on silica gel with 1. ethyl acetate n-hexane 3:2, 2. ethyl acetate.

yield: 0.298 g (59%), yellow solid, solid.t.: 196° C.

Cal.: C 73.64, H, 5.39 N, 8.31; Found: C 73.40, H 5.23, N, 8.26.

IR (KBr): ν=3395, 1680, 1640, 1575, 1520 cm⁻¹. ¹H-NMR (DMSO-d₆): δ=2.25 (s, 3H), 3.36 (s, 2H), 4.04 (m, 2H), 7.00 (m, 5H), 7.02 (m, 2H), 7.47 (m, 5H), 7.60 (m, 2H), 7.65 (m, 2H), 7.79 (m, 1H), 7.87 (m, 2H), 8.68 (m, 1H), 9.99 (s, 1H), 10.11 (s, 1H). ¹³C-NMR (DMSO-d₆): δ=20.4, 42.1, 43.1, 120.3, 122.2, 124.0, 127.1, 127.9, 128.0, 128.7, 129.3, 130.6, 131.0, 131.6, 132.0, 132.3, 133.8, 134.8, 135.2, 137.0, 166.4, 167.7, 168.8, 194.9. MS: m/z=505 (67, M⁺), 212 (100).

### EXAMPLE 26

### N-[3-benzoyl-4-[2-(4-methylphenyl)acetyl]amino]phenyl]naphthalin-1-carboxylic Acid Amide

Steps 1 and 2: s. Example 2

Step 3: N-[3-benzoyl-4-[2-(4-methylphenyl)acetyl]amino]phenyl]benzoic Acid Amide

C₃₃H₂₆N₂O₃ (498.61 gmol⁻¹)

According to general instruction 3 from Naphthylin-1-carboxylic acid chloride (0.19 g, 1.0 mmol) and N-(4-amino-2-benzoylphenyl)-2-(4-methylphenyl) acetamide (0.344 g, 1.0 mmol). Purification: Recrystallization from toluene.

yield: 0.440 g (88%), yellow solid, solid.t.: 208° C.

Cal.: C 79.49, H, 5.27 N, 5.62; Found: C 79.00, H 5.38, N, 5.64.

¹H-NMR (DMSO-d₆): δ=2.26 (s, 3H), 3.37 (s, 2H), 7.00 (m, 2H), 7.05 (m, 2H), 7.48 (m, 2H), 7.58 (m, 5H), 7.69 (m, 3H), 7.88 (m, 1H), 8.03 (m, 3H), 8.18 (m, 1H), 10.05 (s, 1H), 10.61 (s, 1H). ¹³C-NMR (DMSO-d₆): δ=20.4, 42.1, 120.9, 122.8, 124.7, 124.9, 125.3, 126.1, 126.7, 128.0, 128.1, 128.6, 128.7, 129.4, 129.5, 130.0, 132.0, 132.4, 133.0, 134.2, 135.2, 135.3, 137.0, 167.1, 168.9, 194.9. MS: m/z=498 (100, M⁺), 366 (54), 155 (79).

### EXAMPLE 27

### N-[3-benzoyl-4-[2-(4-methylphenyl)acetyl]amino]phenyl]anthracene-9-carboxylic Acid Amide

Steps 1 and 2: s. Example 2

Step 3: N-[3-benzoyl-4-[2-(4-methylphenyl)acetyl]amino]phenyl]benzoic Acid Amide

C₃₇H₂₈N₂O₃ (548.65 gmol⁻¹)

According to general instruction 3 from anthracene-9-carboxylic acid chloride (0.36 g, 1.5 mmol) and N-(4-amino-2-benzoylphenyl)-2-(4-methylphenyl) acetamide (0.516 g, 1.5 mmol). Purification: column chromatography with ethyl acetate n-hexane 2:3. yield: 0.345 g (42%), yellow solid.

¹H-NMR (DMSO-d₆): δ=2.28 (s, 3H), 3.60 (s, 2H), 7.10 (m, 2H), 7.17 (m, 3H), 7.42 (m, 5H), 7.48 (m, 1H), 7.66 (m, 1H), 7.70 (m, 3H), 7.93 (m, 2H), 7.98 (m, 3H), 8.42 (s, 1H), 8.52 (m, 1H), 10.46 (s, 1H).

### EXAMPLE 28

### N-[3-benzoyl-4-[2-(4-methylphenyl)acetyl]amino]phenyl]fluorene-9-carboxylic Acid Amide

Steps 1 and 2: s. Example 2

Step 3: N-[3-benzoyl-4-[2-(4-methylphenyl)acetyl]amino]phenyl]benzoic Acid Amide

C₃₆H₂₈N₂O₃ (536.64 gmol⁻¹)

According to general instruction 3 from fluorene-9-carboxylic acid chloride (0.34 g, 1.5 mmol) and N-(4-amino-2-benzoylphenyl)-2-(4-methylphenyl) acetamide (0.516 g, 1.5 mmol). Purification: Recrystallization from toluene.

yield: 0.475 g (42%), yellow solid.

¹H-NMR (DMSO-d₆): δ=2.22 (s, 3H), 3.32 (s, 2H), 4.96 (s, 1H), 6.95 (m, 2H), 7.01 (m, 2H), 7.29 (m, 2H), 7.37-7.45 (m, 4H), 7.55 (m, 4H), 7.62 (m, 2H), 7.69 (m, 1H), 7.80 (m, 1H), 7.83 (m, 2H), 9.99 (s, 1H), 10.67 (s, 1H).

### EXAMPLE 29

### N-[2-[3-benzoyl-4-[[2-(4-methylphenyl)acetyl]amino]phenyl]benzyl Thioglycolic Acid Amide

Steps 1 and 2: s. Example 2

Step 3: N-[2-[3-benzoyl-4-[[2-(4-methylphenyl)acetyl]amino]phenyl]benzyl Thioglycolic Acid Amide

C₃₁H₂₈N₂O₃S (508.68 gmol⁻¹)

According to general instruction 3 from benzyl thioglycolic acid chloride (0.241 g, 1.2 mmol) and N-(4-amino-2-benzoylphenyl)-2-(4-methylphenyl) acetamide (0.413 g, 1.2 mmol). Purification: MPLC on silica gel with ethyl acetate/n-hexane 1:1. Recryst. from n-hexane/acetone.

yield: 0.341 g (67%), yellow solid, solid.t.: 120° C.

Cal.:C 73.19, H 5.56, N 5.51, S 6.30. Found: C 73.10, H 5.53, N, 5.48, S 6.58.

IR (KBr): ν=3305, 1700, 1680, 1635, 1590, 1510 cm⁻¹. ¹H-NMR (CDCl₃): δ=2.34 (s, 3H), 3.23 (s, 2H), 3.68, (s, 2H), 3.73 (s, 2H), 7.19 (m, 9H), 7.37 (m, 1H), 7.50 (m, 2H), 7.60 (m, 1H), 7.71 (m, 3H), 8.33 (s, 1H), 8.51 (m, 1H), 10.52 (s, 1H). ¹³C-NMR (CDCl₃): δ=21.1, 36.4, 37.6, 45.0, 122.2, 124.1, 124.3, 125.1, 127.6, 128.3, 128.8, 128.8, 129.3, 129.6, 130.0, 131.1, 131.7, 132.8, 136.6, 137.0, 138.0, 166.6, 170.2, 198.5. MS: m/z=508 (100, M⁺), 386 (52), 254 (84), 91 (54).

### EXAMPLE 30

### N-[2-[3-benzoyl-4-[[2-(4-methylphenyl)acetyl]amino]phenyl]-4-benzyloxy Acetic Acid Amide

Steps 1 and 2: s. Example 2

Step 3: N-[2-[3-benzoyl-4-[[2-(4-methylphenyl)acetyl]amino]phenyl]benzyloxy Acetic Acid Amide

C₃₁H₂₈N₂O₄ (492.61 gmol⁻¹)

According to general instruction 3 from benzyloxy acetic acid chloride (0.185 g, 1.0 mmol) and N-(4-amino-2-benzoylphenyl)-2-(4-methylphenyl) acetamide (0.344 g, 1.0 mmol). Purification: MPLC on silica gel with ethyl acetate/n-hexane 1:1.

yield: 0.379 g (77%), brown-yellow resin.

Cal.: C 75.59, H, 5.73 N, 5.69; Found: C 75.40, H 5.85, N, 5.41.

IR (KBr): ν=3305, 3030, 2920, 1685, 1595, 1510 cm⁻¹. ¹H-NMR (CDCl₃): δ=2.34 (s, 3H), 3.69 (s, 2H), 4.04 (s, 2H), 4.62 (s, 2H), 7.17 (m, 2H), 7.24 (m, 2H), 7.35 (m, 5H), 7.49 (m, 2H), 7.54 (m, 1H), 7.60 (m, 1H), 7.71 (m, 2H), 7.89 (m 1H), 8.25 (s, 1H), 8.55 (m, 1H), 10.53 (s, 1H). ¹³C-NMR (CDCl₃): δ=21.1, 45.1, 69.5, 73.9, 122.4, 124.2, 124.5, 125.3, 128.1, 128.4, 128.5, 128.8, 129.3, 129.6, 130.1, 131.2, 131.4, 132.8, 136.3, 136.7, 137.0, 138.1, 167.6, 170.3, 198.5. MS: m/z=492 (100, M⁺), 360 (65), 91(55).

### EXAMPLE 31

### N-[3-benzoyl-4-[2-(4-methylphenyl)acetyl]amino]phenyl]-4-(4-nitrophenyl)butyric Acid Amide

Steps 1 and 2: s. Example 2

Step 3: N-[3-benzoyl-4-[2-(4-methylphenyl)acetyl]amino]phenyl]-4-phenylbutyric Acid Amide

C₃₂H₃₉N₃O₅ (535.61 gmol⁻¹)

According to general instruction 3 from 4-(4-nitrophenyl)butyric acid chloride (0.340 mg, 1.5 mmol) and N-(4-amino-2-benzoylphenyl)-2-(4-methylphenyl) acetamide (0.512 g, 1.5 mmol). Purification: Recrystallization from toluene.

yield: 0.330 g (41%), yellow solid.

¹H-NMR (DMSO-d₆): δ=1.90 (m, 2H), 2.23 (s, 3H), 2.30 (m, 2H), 2.73 (m, 2H), 3.32 (s, 2H), 6.95 (m, 2H), 7.01 (m, 2H), 7.48 (m, 5H), 7.62 (m, 4H), 7.75 (m, 1H), 8.11 (m, 2H), 9.91 (s, 1H), 9.96 (s, 1H).

### EXAMPLE 32

### N-[2-[3-benzoyl-4-[[2-(4-methylphenyl)acetyl]amino]phenyl]-(4-nitrobenzyl)thioglycolic Acid Amide

Steps 1 and 2: s. Example 2

Step 3: N-[2-[3-benzoyl-4-[[2-(4-methylphenyl)acetyl]amino]phenyl]-(4-nitrobenzyl)thioglycolic Acid Amide

C₃₁H₂₇N₃O₅S (553.68 gmol⁻¹)

According to general instruction 3 from 4-nitrobenzylthioglykol acid chloride (0.295 g, 1.2 mmol) and N-(4-amino-2-benzoylphenyl)-2-(4-methylphenyl) acetamide (0.413 g, 1.2 mmol). Purification: MPLC on silica gel with ethyl acetate/n-hexane 1:1.

yield: 0.397 g (60%), yellow solid, solid.t.: 60° C.

Cal.: C 67.24, H 4.93, N 7.59, S 5.79. Found: C 67.04, H 5.08, N, 7.22, S 5.86.

IR (KBr): ν=3295, 1665, 1600, 1555, 1515 cm⁻¹. ¹H-NMR (CDCl₃): δ=2.34, (s, 3H), 3.16, (s, 2H), 3.68, (s, 2H), 3.82 (s, 2H), 7.15 (m, 2H), 7.22 (m, 2H), 7.47 (m, 5H), 7.61 (m, 1H), 7.70 (m, 2H), 7.82 (s, 1H), 8.10 (m, 2H), 8.31 (s, 1H), 8.50 (m, 1H), 10.47 (s, 1H). ¹³C-NMR (CDCl₃): δ=21.1, 35.8, 36.2, 45.0, 122.5, 123.9, 124.1, 124.4, 125.0, 128.4, 129.3, 129.6, 129.8, 130.0, 131.1, 131.8, 132.8, 136.7, 137.1, 137.9, 144.4, 147.2, 166.4, 170.4, 198.3. MS: m/z=553 (0.5, M⁺), 554 (85), 421 (100).

### EXAMPLE 33

### N-[3-benzoyl-4-[(4-methylphenyl)acetylamino]phenyl]-4-propyloxyzimt-acid Amide

Steps 1 and 2: s. Example 2

3. Step: N-[3-benzoyl-4-[(4-methylphenyl)acetylamino]phenyl]-4-propyloxycinnamic Acid Amide

C₃₄H₃₂N₂O₄ (532.68 gmol⁻¹)

According to general instruction 3 from 4-propyloxycinnamic acid chloride (0.270 g, 1.2 mmol) and N-(4-amino-2-benzoylphenyl)-2-(4-methylphenyl) acetamide (0.413 g, 1.2 mmol). Purification: MPLC etOac:n-hexane 1:1.

yield: 0.384 g (60%), light yellow solid, solid.t.: 175° C.

Cal.: C, 76.66; H, 6.07; N, 5.26; Found: C, 76.26; H, 5.88; N, 5.56.

IR (KBr): ν=3230, 3035, 2965, 1665, 1605, 1550, 1515 cm⁻¹. ¹H-NMR (DMSO-D₆): δ=0.96 (t, J=7 Hz, 3H), 1.67-1.77 (m, 2H), 2.26 (s, 3H), 3.37 (s, 2H), 3.97 (t, J=6 Hz, 2H), 6.60 (d, J=16 Hz, 1H), 6.98-6.96 (m, 2H), 7.00-7.02 (m, 2H), 7.04-7.06 (m, 2H), 7.50-7.53 (m, 5H), 7.60-7.70 (m, 4H), 7.77 (s, 1H), 7.88 (m, 1H), 10.02 (s, 1H), 10.16 (s, 1H). MS: m/z 532 (10) [M⁺], 344 (11), 189 (39), 98 (23), 83 (38), 73 (93), 69 (97), 55 (85), 43 (100).

### EXAMPLE 34

### N-[3-benzoyl-4-[(4-bromphenyl)acetylamino]phenyl]-4-propyloxycinnamic Acid Amide

Steps 1 and 2: s. Example 36

3. Step: N-[3-benzoyl-4-[(4-bromphenyl)acetylamino]phenyl]-4-propyloxycinnamic Acid Amide

C₃₃H₂₉BrN₂O₄ (597.51 gmol⁻¹)

According to general instruction 3 from 4-propyloxycinnamic acid chloride (0.103 g, 0.5 mmol) and N-(4-amino-2-benzoylphenyl)-2-(4-bromphenyl) acetamide (0.205 g, 0.5 mmol). Purification: Recrystallization from ethanol.

yield: 0.193 g (65%), light brown pin formed crystals, solid.t.: 178° C.

Cal.: C, 66.34; H, 4.89; N, 4.69; Found: C, 66.31; H, 4.98; N, 4.77.

IR (KBr): ν=3428, 3327, 2968, 2938, 1663, 1603, 1550, 1509, 1400, 1285, 1171, 977 cm⁻¹.

¹H-NMR (CDCl₃): δ=0.96 (t, J=8 Hz, 3H); 1.73 (m, 2H); 3.86 (t, J=7 Hz, 2H); 6.23 (d, J=16 Hz, 1H); 6.80 (m, 2H); 7.16-7.19 (m, 2H); 7.32-7.43 (m, 6H); 7.56 (d, J=16 Hz, 1H); 7.58-7.66 (m, 4H); 7.97 (s, 1H); 8.47 (m, 1H); 10.61 (s, 1H).

MS: m/z=598 (13, M⁺), 189 (100), 147 (44), 44 (32), 55 (26), 91(23), 119 (23), 69 (19), 105 (14), 212 (14).

### EXAMPLE 35

### N-[3-benzoyl-4-[(4-methylphenyl)acetylamino]phenyl]-3-[5-(4-nitrophenyl)-2-furyl]acrylic Acid Amide

Steps 1 and 2: s. Example 2

3. Step: N-[3-benzoyl-4-[(4-methylphenyl)acetylamino]phenyl]-3-[5-(4-nitrophenyl)-2-furyl]acrylic Acid Amide

According to general instruction 3 from 3-[5-(4-nitrophenyl)-2-furyl]acrylic acid chloride (0.333 g, 1.2 mmol) and N-(4-amino-2-benzoylphenyl)-2-(4-methylphenyl) acetamide (0.413 g, 1.2 mmol). Purification: MPLC etOac: n-hexane 2:1.

C₃₅H₂₇N₃O₆ (585.65 gmol⁻¹)

According to general instruction 3 from 3-[5-(4-nitrophenyl)-2-furyl]acrylic acid (g, mmol) and N-(4-amino-2-benzoylphenyl)-2-(4-methylphenyl) acetamide (g, mmol). Purification: MPLC etOac: n-hexane 1:1 and Recrystallization from n-hexane: acetone. yield: 0.311 g (48%), yellow solid, solid.t.: 221° C.

Cal.: C, 71.77; H, 4.65; N, 7.18; Found: C, 71.40; H, 4.69; N, 7.05.

IR (KBr): ν=3260, 2920, 1665, 1630, 1595, 1550, 1510 cm⁻¹. ¹H-NMR (CDCl₃): δ=2.33, (s, 3H), 3.69, (s, 2H), 6.59 (d, J=15 Hz, 1H), 6.67-6.68 (m, 1H), 6.90-6.91 (m, 1H), 7.15-7.17 (m, 2H), 7.23-7.25 (m, 2H), 7.45-7.48 (m, 3H), 7.56-7.59 (m, 1H), 7.61-7.64 (m, 1H), 7.70-7.71 (m, 2H), 7.76 (m, 2H), 7.93 (s, br, 1H), 8.03 (s, br, 1H), 8.20 (m, 2H), 8.51 (m, 1H), 10.52 (s, br, 1H). MS: m/z=585 (52) [M⁺], 586 (20) [M⁺+1], 583 (15), 453 (18), 345 (17), 344 (71), 326 (18), 325 (20), 243 (15), 242 (100), 238 (15), 212 (80), 211 (29), 196 (22), 105 (39), 40 (11).

### EXAMPLE 36

### N-[3-benzoyl-4-[(4-bromphenyl)acetylamino]phenyl]-3-[5-(4-nitrophenyl)-2-furyl]acrylic Acid Amide

1. Step: N-(2-benzoyl-4-nitrophenyl)-2-(4-bromphenyl)acetamide

C₂₁H₁₅BrN₂O₄ (439.27 gmol⁻¹)

According to general instruction 1 from 2-amino-4-nitrobenzophenon (1.2 g, 5.0 mmol) and 2-(4-Bromphenyl)acetylchlorid (1.167 g, 5.0 mmol). Purification: Recrystallization from ethanol.

yield: 1.91 g (87%), yellow crystals, solid.t.: 138° C.

Cal.: C, 57.42; H, 3.44; N, 6.38; Found: C, 57.71; H, 3.73; N, 6.42.

IR (KBr): ν=3427, 1716, 1637, 1614, 1580, 1542, 1508 cm⁻¹. ¹H-NMR (CDCl₃): δ=3.75 (s, 2H), 7.25 (m, 2H), 7.29 (m, 2H), 7.24 (m, 2H), 7.61 (m, 3H), 8.39 (m, 1H), 8.45 (m, 1H), 8.88 (m, 1H), 11.17 (s, 1H). MS: m/z=59 (100), 43 (77), 72 (51), 45 (46), 41 (44), 269 (13), 198 (11), 81 (11), 408 (2), 438 (1) [M⁺], 440 (0.9) [M⁺+2].

2. Step: N-(4-amino-2-benzoylphenyl)-2-(4-bromphenyl)acetamide

C₂₁H₁₇BrN₂O₂ (409.29 gmol⁻¹)

According to general instruction 2 from N-(2-benzoyl-4-nitrophenyl)-2-(4-bromphenyl)acetamide (1.490 g, 3.8 mmol)

yield: 1.124 g (82%), yellow solid, solid.t.: 172° C.

IR (KBr): ν=3437, 3280, 2923, 1661, 1595, 1551, 1531, 1501 cm⁻¹. ¹H-NMR (CDCl₃): δ=3.58 (s, 4H), 6.71 (m, 1H), 6.80 (m, 1H), 7.15 (m, 1H), 7.19 (m, 1H), 7.40 (m, 4H), 7.52 (m, 1H), 7.61 (m, 2H), 8.21 (m, 1H), 10.16 (s, 1H). MS: m/z=212 (100), 408 (70) [M⁺], 410 (70) [M⁺+2], 211 (50), 409 (17), 41 (16), 239 (16), 213 (15), 105 (14), 169 (9), 171 (8), 210 (8).

3. Step: N-[3-benzoyl-4-[(4-bromphenyl)acetylamino]phenyl]-3-[5-(4-nitrophenyl)-2-furyl]-acrylic Acid Amide

C₃₄H₂₄BrN₃O₆ (650.46 gmol⁻¹)

According to general instruction 3 from 3-[5-(4-nitrophenyl)-2-furyl]acrylic acid chloride (0.277 g, 1.0 mmol) and N-(4-amino-2-benzoylphenyl)-2-(4-bromphenyl)acetylamide (0.409 g, 1.0 mmol). Purification: Recrystallization from toluene.

yield: 0.532 g (82%), yellow solid, solid.t.: 237° C.

Cal.: C, 62.78; H, 3.72; N, 6.46; Found: C, 63.08; H, 4.10; N, 6.44.

IR(KBr): ν=3384, 3302, 1680, 1628, 1597, 1553, 1511 cm⁻¹. ¹H-NMR(DMSO-D₆): δ=3.38 (s, 2H), 6.76 (d, J=16 Hz, 1H), 7.03-7.05 (m, 3H), 7.39-7.42 (m, 4H), 7.45-7.48 (m, 2H), 7.54-7.56 (m, 1H), 7.59-7.62 (m, 1H), 7.65-7.67 (m, 2H), 7.76-7.77 (m, 1H), 7.87-7.89 (m, 1H), 7.98-7.99 (m, 2H), 8.29-8.31 (m, 2H), 10.05 (s, 1H), 10.37 (s, 1H). MS: m/z=212 (100), 242 (78), 408 (49), 211 (48), 410 (47), 238 (33), 239 (21), 196 (20), 169 (17), 171 (16), 105 (16), 183 (15) 650 (4).

### EXAMPLE 37

### N-[3-benzoyl-4-[(4-trifluormethylphenyl)acetylamino]phenyl]-3-[5-(4-nitrophenyl)-2-furyl]acrylic Acid Amide

1. Step: N-(2-benzoyl-4-nitrophenyl)-trifluoro acetamide C₁₅H₉F₃N₂O₄ (338.25 gmol⁻¹)

Trifluoro acetic anhydride (0.75 ml, 5.0 mmol; 0.15 ml pro mmol amine) is added dropwise to a solution of 2-amino-4-nitrobenzophenone (1.2 g, 5.0 mmol) into a mixture of dry dichloromethan (8.5 ml pro mmol amine) and dry pyridine (0.9 ml pro mmol amine) with the help of a syringe at 0° C. The reaction mixture is stirred at room temperature for two hours. Subsequently, the solution is diluted with dichloromethan and is successively washed with water, saturated solution of sodium hydrogen carbonate solution and saturated solution of sodium chloride. After drying over sodium sulfate the solvent is distilled in the rotary evaporator. Purification: Recrystallization from ethanol.

yield: 1.420 g (83%), white crystals, solid.t.: 135° C.

Cal.: C, 53.27; H, 2.68; N, 8.28; Found: C, 53.03; H, 2.91; N, 8.28.

IR (KBr): ν=3432, 1735, 1643, 1619, 1587, 1558, 1522 cm⁻¹. ¹H-NMR (CDCl₃): δ=7.56 (m, 2H), 7.71 (m, 3H), 8.50 (m, 1H), 8.57 (m, 1H), 8.87 (m, 1H), 12.27 (s, 1H). MS: m/z=105 (100), 77 (69), 338 (55) [M⁺], 269 (34), 191 (32), 145 (16), 51 (15), 69 (11), 339 (10), 139 (8), 106 (8), 241 (7).

2. Step: N-(4-amino-2-benzoylphenyl)-trifluoroacetamide

C₁₅H₁₁F₃N₂O₂ (308.26 gmol⁻¹)

According to general instruction 2 from N-(2-benzoyl-4-nitrophenyl)-trifluoroacetamide (1.4 g, 4.1 mmol)

yield: 1.205 g (95%), yellow solid, solid.t.: 108° C.

IR (KBr): ν=3445, 3389, 3058, 2975, 2874, 1714, 1653, 1595, 1539 cm⁻¹. ¹H-NMR (CDCl₃): δ=6.83 (m, 1H), 6.87 (m, 1H), 7.43 (m, 2H), 7.55 (m, 1H), 7.65 (m, 2H), 8.32 (m, 1H), 11.47 (s, 1H). MS: m/z=308 (100) [M⁺], 105 (91), 77 (54), 211 (29), 161 (21), 239 (18), 309 (17), 51 (13), 106 (12), 210 (10), 78 (10), 52 (9).

3. Step: N-[3-benzoyl-4-(trifluoroacetylamino)phenyl]-3-[5-(4-nitrophenyl)-2-furyl]acrylic Acid Amide

C₂₈H₁₈F₃N₃O₆ (549.47 gmol⁻¹)

According to general instruction 2 from 3-[5-(4-nitrophenyl)-2-furyl]acrylic acid chloride (0.554 g, 2.0 mmol) and N-(4-amino-2-benzoylphenyl)-trifluoroacetamide (0.616 g, 2.0 mmol). Purification: Recrystallization from toluene.

yield: 1.002 g (90%), yellow solid, solid.t.: 244° C.

Cal.: C, 61.21; H, 3.30; N, 7.65; Found: C, 61.09; H, 3.24; N, 7.31.

IR (KBr): ν=3426, 1730, 1684, 1626, 1599, 1520 cm⁻¹. ¹H-NMR (DMSO-D₆): δ=6.74 (d, J=16 Hz, 1H), 7.06 (m, 1H), 7.38 (m, 2H), 7.47 (m, 3H), 7.61 (m, 1H), 7.64 (m, 2H), 7.83 (m, 1H), 7.95 (m, 3H), 8.27 (m, 2H), 10.53 (s, 1H), 11.24 (s, 1H). MS: m/z=242 (100), 549 (24) [M⁺], 243 (15), 196 (14), 550 (8), 308 (6), 139 (5), 212 (2), 105 (2), 197 (2), 244 (1), 77 (1).

4. Step: N-(4-amino-3-benzoylphenyl)-3-[5-(4-nitrophenyl)-2-furyl]acrylic Acid Amide

C₂₆H₁₉N₃O₅ (453.46 gmol⁻¹)

N-[3-benzoyl-4-(trifluoroacetylamino)phenyl]-3-[5-(4-nitrophenyl)-2-furyl]acrylic acid amide (0.559 g, 1.0 mmol) is heated in a 1:1 mixture (vol:vol) of dioxane and saturated potassium carbonate solution (6 ml per mmol of the protected amine) under reflux for three hours. Subsequently, it is diluted with an equal amount of water and the mixture is extracted with ethyl acetate for three times. The combined organic phases are washed with water and saturated solution of sodium chloride, dried over sodium sulfate and the solvent is distilled of in a rotary evaporator. Purification: Recrystallization from toluene.

yield: 0.385 g (85%), yellow solid, solid.t.: 257° C.

Cal.: C, 68.87; H, 4.22; N, 9.27; Found: C, 68.69; H, 4.44; N, 9.28.

IR (KBr): ν=3487, 1700, 1684, 1622, 1598, 1550, 1511 cm⁻¹. ¹H-NMR (DMSO-D₆): δ=6.70 (d, J=16 Hz, 1H), 6.86 (m, 2H), 6.95 (m, 1H), 7.32 (d, J=16 Hz, 1H), 7.37 (m, 1H), 7.51 (m, 2H), 7.58 (m, 3H), 7.67 (m, 1H), 7.71 (m, 1H), 7.96 (m, 2H), 8.29 (m, 2H), 9.95 (s, 1H). MS: m/z=453 (100) [M⁺], 212 (83), 242 (52), 454 (30), 211 (13), 213 (13), 243 (8), 455 (5), 423 (2), 214 (1), 196 (1), 238 (1).

5. Step: N-[3-benzoyl-4-[(4-trifluormethylphenyl)acetylamino]phenyl]-3-[5-(4-nitrophenyl)-2-furyl]acrylic Acid Amide

C₃₅H₂₄F₃N₃O₆ (639.59 gmol⁻¹)

According to general instruction 1 from N-(4-amino-3-benzoylphenyl)-3-[5-(4-nitrophenyl)-2-furyl]acryl acid amide (0.325 g, 0.75 mmol) and 4-trifluoromethyl phenylacetylchloride (0.166 g, 0.75 mmol). Purification: Recrystallization from toluene.

yield: 0.190 g (40%), orange solid, solid.t.: 235° C.

Cal.: C, 65.73; H, 3.78; N, 6.57; Found: C, 65.42; H, 4.09; N, 6.46.

IR (KBr): ν=3426, 1684, 1626, 1598, 1553, 1511 cm⁻¹. ¹H-NMR (DMSO-D₆): δ=3.52 (s, 2H), 6.77 (d, J=16 Hz, 1H), 7.03 (m, 1H), 7.32 (m, 2H), 7.41 (m, 2H), 7.44 (m, 2H), 7.58 (m, 4H), 7.66 (m, 2H), 7.78 (m, 1H), 7.90 (m, 1H), 7.99 (m, 2H), 8.32 (m, 2H), 10.12 (s, 1H), 10.39 (s, 1H). MS: m/z =43 (100), 55 (86), 41 (84), 57 (77), 59 (69), 69 (56), 44 (54), 73 (50), 285 (38), 239 (35), 129 (32), 639 (1) [M⁺].

### EXAMPLE 38

### N-[3-benzoyl-4-[(4-methylphenyl)acetylaminophenyl]-3-[5-(4-nitrophenyl)-4-thiazolyl]acrylic Acid Amide

Steps 1 and 2: s. Example 2

3. Step: N-[3-benzoyl-4-[(4-methylphenyl)acetylamino]phenyl]-3-[5-(4-nitrophenyl)-4-thiazolyl]acrylic acid amide

C₃₄H₂₆N₄O₅S (602.71 gmol⁻¹)

According to general instruction 3 from 3-[5-(4-nitrophenyl)-4-thiazolyl]acrylic acid chloride (0.354 g, 1.2 mmol) and N-(4-amino-2-benzoylphenyl)-2-(4-methylphenyl)acetamide (0.413 g, 1.2 mmol). Purification: MPLC etOac:n-hexane 2:1.

yield: 0.376 g (52%), yellow solid, solid.t.: 243° C.

Cal.: C, 67.75; H, 4.36; N, 9.30; S, 5.32; Found: C, 67.48; H, 4.46; N, 8.98; S, 5.37.

IR (KBr): ν=3335, 3085, 2920, 1685, 1660, 1635, 1595, 1550, 1515 cm⁻¹. ¹H-NMR (CDCl₃): δ=2.34, (s, 3H), 3.71, (s, 2H), 7.01 (d, J=16 Hz, 1H), 7.17-7.18 (m, 2H), 7.24-7.28 (m, 4H), 7.44-7.54 (m, 3H), 7.59-7.68 (m, 2H), 7.72-7.74 (m, 2H), 8.00 (s, br, 1H), 8.12-8.14 (m, 2H), 8.28-8.32 (m, 2H), 8.57-8.59 (m, 1H), 10.54 (s, br, 1H). MS: m/z=602 (5) [M⁺], 345 (17), 344 (79), 259 (14), 239 (20), 238 (13), 213 (22), 212 (100), 211 (47), 142 (13), 105 (42), 44 (11), 40 (24).

### EXAMPLE 39

### N-[3-benzoyl-4-[(4-methylphenyl)acetylamino]phenyl]-3-[5-(4-bromphenyl)-2-furyl]acrylic Acid Amide

Steps 1 and 2: s. Example 2

3. Step: N-[3-benzoyl-4-[(4-methylphenyl)acetylamino]phenyl]-3-[5-(4-bromphenyl)-2-furyl]acrylic Acid Amide

C₃₅H₂₇BrN₂O₄ (619.54 gmol⁻¹)

According to general instruction 3 from 3-[5-(4-bromophenyl)-2-furyl]acrylic acid chloride (0.355 g, 1.2 mmol) and N-(4-amino-2-benzoylphenyl)-2-(4-methylphenyl) acetamide (0.413 g, 1.2 mmol). Purification: MPLC etOac:n-hexane 1:1.

yield: 0.468 g (63%), yellow solid, solid.t.: 237° C.

Cal.: C, 67.85; H, 4.40; Br, 12.90; N, 4.52; Found: C, 67.79; H, 4.52; Br, 12.69; N, 4.58.

IR (KBr): ν=3300, 3045, 2360, 1685, 1660, 1635, 1595, 1550, 1505 cm⁻¹. ¹H-NMR (CDCl₃): δ=2.33, (s, 3H), 3.68, (s, 2H), 6.43 (d, J=15 Hz, 1H), 6.62-6.64 (m, 1H), 6.68-6.70 (m, 1H), 7.13-7.16 (m, 2H), 7.21-7.25 (m, 3H), 7.30 (s, br, 1H), 7.42-7.62 (m, 8H), 7.71-7.72 (m, 2H), 7.94 (s, br, 1H), 8.54 (d, J=9 Hz, 1H), 10.46 (s, br, 1H). MS: m/z=619 (9) [M⁺], 620 (34) [M⁺+1], 618 (26), 607 (26), 344 (44), 277 (100), 275 (88), 265 (40), 212 (24), 105 (23), 40 (46).

### EXAMPLE 40

### N-[3-benzoyl-4-[(4-methylphenyl)acetylamino]phenyl]-3-[5-(4-chlorphenyl)-2-furyl]acrylic Acid Amide

Steps 1 and 2: s. Example 2

3. Step: N-[3-benzoyl-4-[(4-methylphenyl)acetylamino]phenyl]-3-[5-(4-chlorphenyl)-2-furyl]acrylic Acid Amide

C₃₅H₂₇ClN₂O₄ (575.09 gmol⁻¹)

According to general instruction 3 from 3-[5-(4-chlorophenyl)-2-furyl]acrylic acid chloride (0.321 g, 1.2 mmol) and N-(4-amino-2-benzoylphenyl)-2-(4-methylphenyl) acetamide (0.413 g, 1.2 mmol). Purification: MPLC etOac:n-hexane 1:1.

yield: 0.493 g (71%), yellow solid, solid.t.: 240° C.

Cal.: C, 73.10; H, 4.73; Cl, 6.16; N, 4.87; Found: 72.92; H, 4.82; Cl, 6.00; N, 4.96.

IR (KBr): ν=3320, 1685, 1660, 1635, 1595, 1555, 1505 cm⁻¹. ¹H-NMR (DMSO-D₆): δ=2.26, (s, 3H), 3.38, (s, 2H), 6.70 (d, J=15 Hz, 1H), 6.94-6.95 (m, 1H), 7.00-7.02 (m, 2H), 7.04-7.06 (m, 2H), 7.12-7.13 (m, 1H), 7.38 (d, J=15 Hz, 1H), 7.48-7.55 (m, 4H), 7.62-7.65 (m, 2H), 7.69-7.70 (m, 2H), 7.77-7.79 (m, 3H), 7.88-7.90 (m, 1H), 10.03 (s, br, 1H), 10.30 (s, br, 1H). MS: m/z=575 (29) [M⁺], 576 (29) [M⁺+1], 577 (10) [M⁺+2], 574 (72), 345 (12), 344 (48), 233 (33), 232 (15), 231 (100), 212 (16).

### EXAMPLE 41

### N-[3-benzoyl-4-[[2-(4-methyl-1-piperazinyl)-2-phenylacetyl]amino]phenyl]-3-[5-(4-nitrophenyl)-2-furyl]acrylic Acid Amide

1. Step: N-(2-benzoyl-4-nitrophenyl)-2-chlor-2-phenyl acetamide

C₂₁H₁₅ClN₂O₄ (394.82 gmol⁻¹)

According to general instruction 1 from 2-amino-4-nitrobenzophenon (1.2 g, 5.0 mmol) and 2-Chlor-2-phenylacetylchlorid (0.945 g, 5.0 mmol). Purification: Recrystallization from ethanol.

yield: 1.658 g (84%), yellow crystals, solid.t.: 124° C.

Cal.: C, 63.89; H, 3.83; N, 7.10; Found: C, 63.80; H, 3.89; N, 7.45.

IR (KBr): ν=3421, 1691, 1653, 1644, 1616, 1579, 1539, 1507 cm⁻¹. ¹H-NMR (CDCl₃): δ=5.44 (s, 1H), 7.32 (m, 3H), 7.49 (m, 4H), 7.63 (m, 1H), 7.66 (m, 2H), 8.35 (m, 1H), 8.44 (m, 1H), 8.81 (m, 1H), 12.01 (s, 1H). MS: m/z=269 (100), 270 (18), 191 (13), 105 (4), 118 (2), 271 (2), 192 (1), 394 (1) [M⁺], 223 (1), 125 (1), 145 (1), 226 (1).

2. Step: N-(2-benzoyl-4-nitrophenyl)-2-(4-methyl-1-piperazinyl)-2-phenyl Acetamide

C₂₆H₂₆N₄O₄ (458.52 gmol⁻¹)

N-(2-benzoyl-4-nitrophenyl)-2-chlor-2-phenyl acetamide (0.945 g, 2.4 mmol) is dissolved in 40 ml of acetonitrile and after addition of three equivalents of methylpiperazin (80 ml, 7.2 mmol) the reaction mixture is heated until boiling for 20 hours. After removal of acetonitrile in the rotary evaporator the solid is taken up in dichloromethane and washed with a solution of potassium carbonate, subsequently dried over sodium sulfate and the solvent is removed in the rotary evaporator. Purification: Recrystallization from ethanol. [J. Med. Chem. 23, (1980), 721]

yield: 0.693 g (63%), reddish solid, solid.t.: 78° C.

Cal.: C, 68.11; H, 5.72; N, 12.22; Found: C, 67.93; H, 6.09; N, 12.19.

IR (KBr): ν=3425, 1698, 1641, 1612, 1597, 1578, 1525, 1502 cm⁻¹. ¹H-NMR (CDCl₃): δ=2.26 (s, 3H), 2.53 (s, 8H), 4.01 (s, 1H), 7.29 (m, 3H), 7.35 (m, 2H), 7.55 (m, 2H), 7.68 (m, 1H), 7.77 (m, 2H), 8.34 (m, 1H), 8.43 (m, 1H), 8.85 (m, 1H), 12.09 (s, 1H). MS: m/z=189 (100), 40 (52), 59 (20), 91 (20), 44 (17), 105 (16), 70 (15), 190 (14), 57 (13), 41 (11), 77 (9), 458 (5) [M⁺].

3. Step: N-(4-amino-2-benzoylphenyl)-2-(4-methyl-1-piperazinyl)-2-phenyl acetamide

C₂₆H₂₈N₄O₂ (428.54 gmol⁻¹)

According to general instruction 2 from N-(2-benzoyl-4-nitrophenyl)-2-(4-methyl-1-piperazinyl)-2-phenyl acetamide (0.553 g, 1.2 mmol). yield: 0.439 g (85%), reddish oily substance.

IR (KBr): ν=3369, 2945, 2833, 1712, 1634, 1616, 1597, 1512 cm⁻¹. ¹H-NMR (CDCl₃): δ=2.00 (s, 8H), 2.65 (s, 3H), 3.98 (s, 1H), 6.75 (m, 1H), 6.82 (m, 1H), 7.24 (m, 3H), 7.29 (m, 2H), 7.45 (m, 2H), 7.56 (m, 1H), 7.67 (m, 1H), 8.26 (m, 1H), 11.39 (s, 1H). MS: m/z=189 (100), 91 (27), 190 (25), 70 (22), 44 (19), 59 (19), 43 (17), 105 (16), 57 (14), 55 (13), 43 (13), 428 (10) [M⁺].

4. Step: N-[3-benzoyl-4-[[2-(4-methyl-1-piperazinyl)-2-phenylacetyl]amino]phenyl]-3-[5-(4-nitrophenyl)-2-furyl]acrylic Acid Amide

C₃₉H₃₅N₅O₆ (669.74 gmol⁻¹)

According to general instruction 3 from 3-[5-(4-nitrophenyl)-2-furyl]acrylic acid chloride (0.277 g, 1.0 mmol) and N-(4-amino-2-benzoylphenyl)-2-(4-methyl-1-piperazinyl)-2-phenyl acetamide (0.428 g, 1.0 mmol). Purification: Recrystallization from toluene.

yield: 0.152 g (23%), yellow solid, solid.t.: 160° C.

Cal.: C, 69.94; H, 5.27; N, 10.46; Found: C, 70.07; H, 5.60; N, 10.43.

IR (KBr): ν=3426, 1682, 1630, 1598, 1547, 1510 cm⁻¹. ¹H-NMR (DMSO-D₆): δ=2.28 (s, 8H), 2.71 (s, 3H), 4.19 (s, 1H), 6.79 (d, J=16 Hz, 1H), 7.02-7.03 (m, 1H), 7.11-7.17 (m, 2H), 7.21-7.25 (m, 4H), 7.31-7.33 (m, 4H), 7.40-7.41 (m, 2H), 7.53-7.56 (m, 2H), 7.75-7.77 (m, 2H), 7.98-8.00 (m, 2H), 8.30-8.32 (m, 2H), 10.51 (s, 1H), 10.88 (s, 1H). MS: m/z=189 (100), 190 (15), 105 (8), 91 (7), 77 (4), 70 (3), 212 (3), 42 (3), 44 (2), 43 (2), 428 (2), 58 (2).

The activity of substances is determined in a test system. This system is based on the measuring of the inhibition of growth of parasites, bacteria, viruses and fungi in vitro.

To determine the anti-malaria activity, for example, the inhibition of the growth of malaria parasites in blood cultures is determined.

Some of the micro-organism which should be investigated can only be investigated in animal models. In this case we will use the corresponding model.

Substances which demonstrate an efficacy in the in vitro measuring system will be further investigated in in vivo models. The anti-parasitic, antiviral, fungicidal or anti-bacterial activity will be further evaluated in the appropriate animal model.

### Example 42 Concerning Activity

The antimalarial activity of substances 1 to 32 was determined using in vitro cultures of the causative organism of malaria Plasmodium falciparum. 200 μl of a asynchronous Plasmodium falciparum culture with a 0.4% parasitemia and 2% haematocrit were loaded into each of the wells of a 96 well microtitre plate. A serial dilution series of the compounds was then prepared at concentrations of 100, 10 and 1 μmol l⁻¹. The plates are incubated at 37° C., 3% CO₂ and 5% O₂ over a period of 48 hours. 30 μl of medium supplemented with 27 μCi ml⁻¹ [³H]-hypoxanthine were then added to each well. After 24 hours' incubation, the parasites were harvested by filtration through glass fiber filters and the incorporated radioactivity was measured. Inhibition of parasite growth was measured as the percentage inhibition of tritium incorporation relative to a comparison without substance. The table states the percentage inhibition in dependence of the concentrations.

TABLE I

| | | % Inhibition at | | |
|---|---|---|---|---|
| | | 100 μM | 10 μM | 1 μM |
| example 1 | 2-[N-[3-[3-benzoyl-4-(2-phenylpropionyl)amino]- | 89 | 19 | — |
| | phenylamino]carbamoyl]acetic acid methylester | | | |
| example 2 | 2-[N-[3-[3-benzoyl-4-[[2-(4-methylphenyl)acetyl]- | 95 | 19 | — |
| | amino]phenylamino]carbamoyl]acetic acid methylester | | | |
| example 3 | 3-[N-[3-[3-benzoyl-4-[[2-(4-methylphenyl)acetyl]- | 38 | 0 | — |
| | amino]phenylamino]carbamoyl]propionic acid | | | |
| example 4 | 3-[N-[3-[3-benzoyl-4-[[2-(4-methylphenyl)acetyl]- | 29 | 0 | — |
| | amino]phenylamino]carbamoyl]propionic acid- | | | |
| | methylester | | | |
| example 5 | 4-[N-[3-[3-benzoyl-4-[[2-(4-methylphenyl)acetyl]- | 59 | 0 | — |
| | amino]phenylamino]carbamoyl]butyric acid | | | |
| example 6 | 4-[N-[3-[3-benzoyl-4-[[2-(4-methylphenyl)acetyl]- | 98 | 0 | — |
| | amino]phenylamino]carbamoyl]butyric acid methyles- | | | |
| | ter | | | |
| example 7 | N-[2-[3-benzoyl-4-[[2-(4-methylphenyl)acetyl]- | 83 | 0 | — |
| | amino]phenylamino]-2-oxoethyl]heptadecanoic acid | | | |
| | amide | | | |
| example 8 | N-[4-[3-benzoyl-4-[[2-(4-methylphenyl)acetyl]- | 93 | 0 | — |
| | amino]phenylamino]-4-oxobutyl]pentadecanoic acid | | | |
| | amide | | | |
| example 9 | 4-[N-[3-[3-benzoy1-4-[[2-(4-methylphenyl)acetyl]- | 96 | 0 | — |
| | amino]phenylamino]carbamoyl]butyric acid-N- | | | |
| | tetradecylamid | | | |
| example | N-[3-benzoyl-4-[[2-(4-methylphenyl)acetyl]- | 99 | 0 | — |
| 10 | amino]phenyl]eicosanoic acid amide | | | |
| example | N-[3-[3-benzoyl-4-[(2-phenylacetyl)amino]- | 98 | 0 | — |
| 11 | phenylamino]-3-oxopropyl]hexadecanoic acid amide | | | |
| example | N-[2-[3-benzoyl-4-[[2-(4-methylphenyl)acetyl]- | 89 | 67 | — |
| 12 | amino]phenylamino]-2-oxoethyl]-4-phenylcinnamic | | | |
| | acid amide | | | |
| example | N-[3-[3-benzoyl-4-[(2-phenylacetyl)amino]- | 95 | 97 | — |
| 13 | phenylamino]-3-oxopropyl]-4-benzyloxycinnamic acid | | | |
| | amide | | | |
| example | N-[3-benzoyl-4-[2-(4-methylphenyl)acetyl]amino]- | 34 | 0 | — |
| 14 | phenyl]nicotinic acid amide | | | |
| example | N-[3-benzoyl-4-[2-(4-methylphenyl)acetyl]amino]- | 96 | 77 | 0 |
| 15 | phenyl]benzoic acid amide | | | |
| example | N-[3-benzoyl-4-[2-(4-methylphenyl)acetyl]amino]- | 94 | 0 | 0 |
| 16 | phenyl]-2-phenylacetic acid amide | | | |
| example | N-[3-benzoyl-4-[2-(4-methylphenyl)acetyl]amino]- | 94 | 91 | 0 |
| 17 | phenyl]-3-phenylpropionic acid amide | | | |
| example | N-[3-benzoyl-4-[2-(4-methylphenyl)acetyl]amino]- | 98 | 98 | 9 |
| 18 | phenyl]-3-cyclohexylpropionic acid amide | | | |
| example | N-[3-benzoyl-4-[2-(4-methylphenyl)acetyl]amino]- | 97 | 95 | 0 |
| 19 | phenyl]-4-phenylbutyric acid amide | | | |
| example | N-[3-benzoyl-4-[2-(4-methylphenyl)acetyl]amino]- | 97 | 94 | 0 |
| 20 | phenyl]-5-phenylvaleric acid amide | | | |
| example | N-[3-benzoyl-4-[2-(4-methylphenyl)acetyl]amino]- | 94 | 95 | 0 |
| 21 | phenyl]cinnamic acid amide | | | |
| example | N-[3-benzoyl-4-[2-(4-methylphenyl)acetyl]amino]- | 88 | 82 | 0 |
| 22 | phenyl]-4-nitrocinnamic acid amide | | | |
| example | N-[3-[3-benzoyl-4-[2-(4-methylphenyl)acetyl]amino]- | 94 | 94 | 82 |
| 23 | phenylamino]-4-phenylcinnamic acid amide | | | |
| example | N-[2-[3-benzoyl-4-[[2-(4-methylphenyl)acetyl]- | 97 | 96 | 85 |
| 24 | amino]phenyl]-4-benzyloxycinnamic acid amide | | | |
| example | N-[2-[3-benzoyl-4-[[2-(4-methylphenyl)acetyl]- | 86 | 0 | 0 |
| 25 | amino]phenylamino]-2-oxoethyl]-4-benzoic acid-amid | | | |
| example | N-[3-benzoyl-4-[2-(4-methylphenyl)acetyl]amino]- | 93 | 65 | 0 |
| 26 | phenyl]naphthalin-1-carboxylic acid amide | | | |
| example | N-[3-benzoyl-4-[2-(4-methylphenyl)acetyl]amino]- | | | |
| 27 | phenyl]anthracen-9-carboxylic acid amide | | | |
| example | N-[3-benzoyl-4-[2-(4-methylphenyl)acetyl]amino]- | 98 | 0 | 0 |
| 28 | phenyl]fluoren-9-carboxylic acid amide | | | |
| example | N-[2-[3-benzoyl-4-[[2-(4-methylphenyl)acetyl]- | 97 | 91 | 0 |
| 29 | amino]phenyl]benzyl thioglycolic acid amide | | | |
| example | N-[2-[3-benzoyl-4-[[2-(4-methylphenyl)acetyl]- | 97 | 93 | 0 |
| 30 | amino]phenyl]-4-benzyloxyacetic acid amide | | | |
| example | N-[3-benzoyl-4-[2-(4-methylphenyl)acetyl]amino]- | 96 | 93 | 0 |
| 31 | phenyl]-4-(4-nitrophenyl)butyric acid amide | | | |
| example | N-[2-[3-benzoyl-4-[[2-(4-methylphenyl)acetyl]- | 96 | 0 | 0 |
| 32 | amino]phenyl]-(4-nitrobenzyl)thio glycolic acid amide | | | |

1. Use of at least one 2-phenylene diamine derivative of general formula (I): 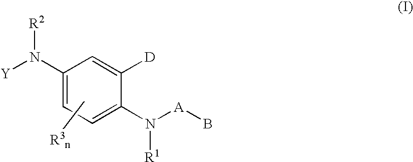
wherein
n=0-3;
R¹, R²=H, C₁₋₂₆-alkyl, aryl, heteroaryl, C₁₋₂₆-acyl;
R³=H, halogen, C₁₋₂₆-alky, aryl, heteroaryl, ar-C₁₋₂₆-alkyl, C₁₋₂₆-acyl, CN, NO₂, R⁴—X—;
with R⁴=H, C₁₋₂₆-alkyl, aryl, heteroaryl, ar-C₁₋₂₆-alkyl, C₁₋₂₆-acyl;
X=NH, O, S, SO₂, NHSO₂, OSO₂,
A=CH₂, CHR⁵, CR⁵R⁶, CO, CS, CONR⁴, CSNR⁴, SO₂, PO₂,
R⁵, R⁶=independently of each other C₁₋₂₆-alkyl, aryl, heteroaryl, ar-C₁₋₂₆-alkyl, CN, NO₂, COR⁷,
R⁷=H, C₁₋₂₆-alkyl, aryl, ar-C₁₋₂₆-alkyl, C₁₋₂₆-alkoxy, aryloxy, ar-C₁₋₂₆-alkoxy, NR⁸R⁹,
R⁸, R⁹=independently of each other H, C₁₋₂₆-alkyl, aryl, ar-C₁₋₂₆-alkyl, heteroaryl,
B=C₁₋₂₆-alkyl, aryl, heteroaryl, arylcarbonyl, saturated heterocycles, substituted alkyl having 1-4 chain members, wherein the substituents may be Cl-g-alkyl, aryl, heteroaryl, halogen, ═O, OH, NH₂, NH—CO—R¹⁰, NH—SO₂—R¹⁰, COOR¹¹, CO—NR¹²R¹³, CS—NR¹⁴R¹⁵, SO₂OR¹⁶, SO₂NR¹⁷R¹⁸, NH—CO—OR¹⁹, NH—CO—NR²OR²¹, NHCSNR²²R²³, R¹⁰-R²³=independently of each other H, C₁₋₂₆-alkyl, aryl, ar-C₁₋₂₆-alkyl, heteroaryl,
D=H, C₁₋₂₆-alkyl, aryl, heteroaryl, ar-C₁₋₂₆-alkyl, —Y—R²⁴, halogen, NO₂, CN, NH—CO—R²⁵, NH—SO₂—R²⁶, NH—CO—OR²⁷, NH—CO—NR²⁸R²⁹, NH—CS—NR³OR³¹,
Y=O, NH, S, CO, CS, SO₂, COO, CONR³¹, CSNR³², SO₂NR³³,
R²⁴-R³³=independently of each other H, C₁₋₂₆-alkyl, aryl, heteroaryl, ar-C₁₋₂₆-alkyl, and
Y=a group selected from 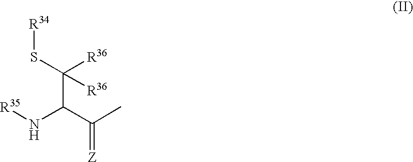
wherein
Z=O, S or two hydrogen atoms,
R³⁴=H, C₁₋₂₆-alkyl, aryl, ar-C₁₋₂₆-alkyl, COOR³⁷, arylsulfonyl,
R³⁵=H, C₁₋₂₆-acyl, COOR³⁸,
R³⁶=independently of each other H, C₁₋₂₆-alkyl,
R³⁷, R³⁸=independently of each other C₁₋₂₆-alkyl, aryl, ar-C₁₋₂₆-alkyl, or 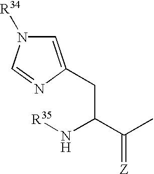
wherein
Z=O, S or two hydrogen atoms,
R³⁴=H, C₁₋₂₆-alkyl, aryl, ar-C₁₋₂₆-alkyl, COOR³⁷, arylsulfonyl,
R³⁵=H, C₁₋₂₆-acyl, COOR³⁸
R³⁷, R³⁸=independently of each other C₁₋₂₆-alkyl, aryl, ar-C₁₋₂₆-alkyl, or 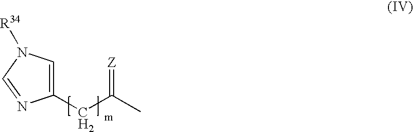
wherein
Z=O, S or two hydrogen atoms,
m=0-3
R³⁴=H, C₁₋₂₆-alkyl, aryl, ar-C₁₋₂₆-alkyl, COOR³⁷, aryl sulfonyl,
R³⁷=C₁₋₂₆-alkyl, aryl, ar-C₁₋₂₆-alkyl, or 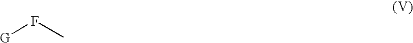
wherein
F=CH₂, CO, CS, SO₂,
G=COOR³⁹, CONHOH, CONR⁴⁰R⁴¹, CSNR⁴²R⁴³, C₁₋₂₆-alkyl- or alkyl substituted by aryl and having 1-3 chain members or alkyl having 1-3 chain members, which supports a substituent at the terminal C-atom the substituent selected from COOR⁴⁴, CONHOH, CONR⁴⁵R⁴⁶, CSNR⁴⁷R⁴⁸, SR⁴⁹, SOR⁵⁰, SO₂R⁵¹, SO₂NR⁵²R⁵³, PO(OR⁵⁴)OR⁵⁵, PO(OR⁵⁶)NR⁵⁷₂, OSO₂R⁵⁸, O(PO)OR⁵⁹, NHSO₂R⁶⁰, NHPO₂R⁶¹, NHCOR⁶², NHCSR⁶³, NHCONR⁶⁴R⁶⁵, NHCSNR⁶⁶R⁶⁷, —S(NH)₂—R⁶⁸ or NH(C═NR⁷⁰)NHR⁶⁹, furthermore aryl or heteroaryl,
R³⁹-R⁶⁹=independently of each other H, C₁₋₂₆-alkyl, aryl,
R⁷⁰=CONH₂, SO₂NH₂, or is 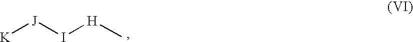
wherein
H=CH₂, CO, CS, CHR⁷¹, CR⁷²R⁷³, SO₂, SO, PO₂,
I=C₁₋₂₆-alkylene, C₁₋₂₆-alkylene, in which one methylene group is replaced by O, S, or NR⁷⁷, or C₂₋₂₆-alaenylene, these alkylene or alkenylene radicals being unsubstituted or substituted by aryl, heteroaryl, halogen, OH, CN, C₁₋₉-alkyloxy, aryloxy, COOR⁷⁴, CONR⁷⁵R⁷⁶, NR⁷⁷R⁷⁸, NH(C═NR⁷⁰)NHR⁶⁹, SR⁷⁹, SO₂R⁸⁰; furthermore C₃₋₈-cycloalkylene, unsubstituted or substituted by aryl, heteroaryl, halogen, OH, CN, C₁₋₉-alkyloxy, aryloxy, COOR⁷⁴, CONR⁷⁵R⁷⁶, NR⁷⁷R⁷⁸, SR⁷⁹, SO₂R⁸⁰; C₃₋₈-cycloalkylene, wherein the alkylene chain is interrupted by O, S or NR⁷⁷, arylene, unsubstituted or substituted by aryl, heteroaryl, halogen, OH, CN, C₁₋₂₆-alkoxy, aryloxy, COOR⁷⁴, CONR⁷⁵R⁷⁶, NR⁷⁷R⁷⁸, SR⁷⁹, SOR⁸⁰,
R⁷¹-R⁸⁰=independently of each other H, C₁₋₂₆-alkyl, aryl, ar-C₁₋₂₆-alkyl, heteroaryl,
J=a bonding or CO, COO, CONR⁸¹, CS, CSNR⁸², SO₂, S(NH)₂, SO(NH), SO₂O, SO₂NR⁸³, PO(OR⁸⁴), PO(OR⁸⁵)NR⁸⁶, NR⁸⁷CO, NR⁸⁸CS, NR⁸⁹SO₂, OSO₂, NR⁹⁰PO(OR⁸⁴), OPO(OR⁹¹), PO(OR⁸⁴)O, NR⁹²CONR⁹³, NR⁹⁴CSNR⁹⁵, NR⁹⁶SO₂NR⁹⁷, NR⁹⁸C(NR⁹⁹)NR¹⁰⁰,
R⁸¹-R¹⁰⁰=independently of each other H, C₁₋₂₆-alkyl, aryl, ar-C₁₋₂₆-alkyl, heteroaryl,
R⁹⁹=H, C₁₋₂₆-alkyl, aryl, ar-C₁₋₂₆-alkyl, heteroaryl, CONR¹⁰¹R¹⁰², CSNR¹⁰³R¹⁰⁴, SO₂NR¹⁰⁵R¹⁰⁶
R¹⁰¹-R¹⁰⁶=independently of each other H, C₁₋₂₆-alkyl, aryl, ar-C₁₋₂₆-alkyl, heteroaryl,
K=branched or unbranched C₁₁₋₂₃-alkyl, branched or unbranched C₁₁₋₂₃-alkenyl being unsubstituted or substituted by aryl or heteroaryl, C₁₁₋₂₃-alkinyl, aryl, heteroaryl, ar-C₁₋₂₆-alkyl, wherein aryl, heteroaryl and ar-C₁₋₂₆-alkyl are substituted by further aryl-, heteroaryl- and/or ar-C₁₋₂₆-alkyl radicals,
or one of its salts for the therapeutic and prophylactic treatment of infections.

2. Use according to claim 1, characterized in that the following is valid for the compounds of formula (I) gilt:
n=0-3,
R¹, R²and R³=H,
A=CO, SO₂,
B=phenyl, benzyl, phenethyl, 4-chlorophenylmethyl, 4-bromophenylmethyl, 4-nitrophenylmethyl, 4-trifluoromethylphenylmethyl, 4-methylphenylmethyl, 1-(4-methyl-1-piperazinyl)-1-(4-trifluoromethylphenyl)methyl, 1-(4-methyl-1-piperazinyl)-1-phenylmethyl, 4-phenylphenyl-methyl, 1-naphthylmethyl, 2-naphthylmethyl, benzoyl, 2,4,4-trimethylpentyl, 2-carboxyethyl, 3-carboxypropyl,
D=benzoyl,
Y=
a radical of formula (II),
wherein
Z=O,
R³⁴=H, benzyloxycarbonyl, trityl,
R³⁵=H, benzyloxycarbonyl, tert.-butyloxycarbonyl,
R³⁶=H.
or a radical of formula (III),
wherein
Z=O,
R³⁴=H, tosyl, benzyl, 4-nitrobenzyl, 4-cyanobenzyl, benzyloxycarbonyl,
R³⁵=H, benzyloxycarbonyl, tert.-butyloxycarbonyl,
or a radical of formula (IV),
wherein
Z=O,
m=0-3,
R³⁴=H, tosyl, benzyl, 4-nitrobenzyl, 4-cyanobenzyl, benzyloxycarbonyl,
or a radical of formula (V),
wherein
F=CO, CH₂, SO₂,
G=COOH, COOMe, CONHOH, CH₂—COOH, CH₂COOMe, CH₂CONHOH, CH₂CONH—(C₁₄-C₂₀)-alkyl, CH₂CH₂COOH, CH₂CH₂COOMe, CH₂CH₂CONHOH, CH₂CH₂CONH—(C_{14-C}₂₀)-alkyl, CH₂CH₂CH₂COOH, CH₂CH₂CH₂COOMe, CH₂CH₂CH₂CONHOH, CH₂CH₂CH₂CONH—(C₁₄-C₂₀)-alkyl, phenyl, naphthyl, pyrridyl, fluorenyl, anthracenyl,
or a radical of formula (VI),
wherein
H=CO, SO₂,
I=methylene, 1,2-ethylene, 1,3-trimethylene, 1,4-tetramethylene, —CH₂—S—CH₂—, —CH₂—O—CH₂—, —CH₂—NH—CH₂—, 1,2-ethenylene, 1,1-ethylene, prop-1-en-1,3-ylene, prop-2-en-1,3-ylene, benzylene, 2-phenyl-1,1-ethylene, carboxymethylene, amino carbonyl methylene, 2-Carboxy-1,1-ethylene, 2-aminocarbonyl-1,1-ethylene, 3-carboxy-1,1-propylene, 3-aminocarbonyl-1,1-propylene, 2-methyl-1,1-propylene, 3-methyl-1,1-butylene, 2-methyl-1,1-butylene, pyrrolidine connected via N1 and C2, 1,2-phenylene, 1,3-phenylene, 1,2-naphthylene, 1,3-naphthylene, 1,1-cyclopentylene, 1,1-cyclohexylene, 1,2-cyclohexylene,
J=a bonding, CO, CS, SO₂, PO(OMe), PO(OH), CONH, CSNH, SO₂NH, PO(OH)O, PO(OH)NH, PO(OMe)O, PO(OMe)NH,
K=(C₁₃-C₁₉)-alkyl, (C₁₃-C₁₉)-alkenyl, 4-benzyloxystyryl, 4-styrylstyryl, 4-phenylstyryl, 4-cyanostyryl, 4-nitrostyryl, phenyl, 4-biphenylyl, 4-nitrophenyl, 4-cyanophenyl, 4-methylsulfonylphenyl, 4-methoxyphenyl, 4-bromophenyl, 4-chlorophenyl, 4-trifluoromethylphenyl, 4-Formylphenyl, 4-methoxycarbonylphenyl, 4-(1,1-dicyano-2-vinyl)phenyl, 4-aminophenyl, 4-ethylphenyl, 4-Isopropylphenyl, 4-tert.-butylphenyl, 4-ethoxyphenyl, 4-propoxyphenyl, 4-butoxyphenyl, 4-benzyloxyphenyl, 3,4-bis-(benzyloxy)phenyl 3-phenoxyphenyl, 4-styrylphenyl 1-naphthyl, 2-naphthyl, 2-fluorenyl, 2-(2-phenylthiazol-4-yl), 5-(4-nitrophenyl)thiazo-4-yl) 5-(4-nitrophenyl)-furan-2-yl), 5-(3-nitrophenyl)furan-2-yl), 5-(2-nitrophenyl)furan-2-yl), 5-(4-bromophenyl)furan-2-yl), 5-(4-chlorophenyl)furan-2-yl), 5-(3-trifluoromethylphenyl)-furan-2-yl), 5-(4-trifluoromethylphenyl)furan-2-yl), 5-(2-chloro-3-trifluoromethylphenyl)-furan-2-yl) 3,4-methylenedioxyphenyl, 1-acetylindol-3-yl, 4′-nitrobiphenylyl, 5-(4-bromophenyl)thiophen-2-yl), 5-(4-methylphenyl)furan-2-yl), 5-(4-methoxyphenyl)furan-2-yl), 5-bromothiophen-2-yl, 5-bromofuran-2-yl, 4-pyridyl, 3-pyridyl, 2-pyridyl, Chinolin-2-yl, 1-naphthylvinyl, 2-naphthylvinyl, 2-fluorenyl-vinyl, 2-(2-phenylthiazol-4-yl)vinyl, 2-[5-(4-nitrophenyl)furan-2-yl)vinyl, 2-[5-(4-acetoxy-methylphenyl)furan-2-yl)vinyl, 2-[5-(3-trifluoromethylphenyl)furan-2-yl)vinyl, 4-benzyloxy-styryl, 3,4-dibenzyloxystyryl, 3-methoxy-4-(4-nitrobenzyloxy)styryl, 2-methylindol-3-ylvinyl, 1acetylindol-3-ylvinyl, 3,4-methylenedioxystyryl, 4-(1,1 dicyano-2-vinyl)styryl and the salts thereof.

3. Use according to claim 1, characterized in that the following is valid for the compounds of formula (I):
n=0-3,
R¹, R²=H
R³H,
A=CO, SO₂,
B=phenyl, benzyl, phenethyl, 4-chlorophenylmethyl, 4-methylphenylmethyl, 1-(4-methyl-1-piperazinyl)-1-(4-trifluoromethylphenyl)methyl, 1-(4-methyl-1-piperazinyl)-1-phenylmethyl, 4-bromophenylmethyl, 4-nitrophenylmethyl, 4-trifluoromethylphenylmethyl, 4-phenylphenyl-methyl, 1-naphthylmethyl, 2-naphthylmethyl, benzoyl, 2,4,4-trimethylpentyl, 2-Carboxyethyl, 3-Carboxypropyl,
D=benzoyl,
Y=
a radical of formula (IV)
wherein
Z=O,
m=0-3,
R³⁴=H, benzyl, 4-nitrobenzyl, 4-cyanobenzyl,
or a radical of formula (V)
wherein
F=CO, CH₂ SO₂,
G=CH₂CH₂CONR—(C₁₄-C₁₈)-alkyl, CH₂CH₂CH₂CONH—(C₁₄-C₁₈)-alkyl, phenyl, naphthyl, pyridyl, fluorenyl, anthracenyl
or a radical of formula (VI)
H=CO,
I=methylene, 1,2-ethylene, 1,3-trimethylene, 1,4-tetramethylene, —CH₂—S—CH₂—, —CH₂—O—CH₂—, —CH₂—NH—CH₂—, 1,2-ethenylene, 1,1-ethylene, prop-1-en-1,3-ylene, prop-2-en-1,3-ylene, benzylene, 2-phenyl-1,1-ethylene, 2-methyl-1,1-propylene, 3-methyl-1,1-butylene, 2-methyl-1,1-butylene, pyrrolidine connected via N1 and C2,
J=PO(OMe), PO(OH), CONH, CSNH, SO₂NH, PO(OH)O, PO(OH)NH, PO(OMe)O, PO(OMe)NH,
K=(C₁₃-C₁₉)-alkyl, (C₁₃-C₁₉)-alkenyl, 4-benzyloxystyryl, 4-phenylstyryl, 4-cyanostyryl, 4-nitrostyryl, phenyl, 4-biphenylyl, 4-nitrophenyl, 4-cyanophenyl, 4-methoxyphenyl, 1-naphthylvinyl, 2-naphthylvinyl, 2-fluorenylvinyl, 2-(2-phenylthiazol-4-yl)vinyl, 2-[5-(4-nitrophenyl)furan-2-yl)vinyl, 2-[5-(4-acetoxymethylphenyl)furan-2-yl)vinyl, 2-[5-(3-trifluoromethylphenyl)furan-2-yl)vinyl, 3,4-dibenzyloxystyryl, 3-methoxy-4-(4-nitrobenzyloxy)styryl, 3,4-methylenedioxystyryl, 4-(1,1 dicyano-2-vinyl)styryl and the salts thereof.

4. Use according to one of claims 1 to 4, characterized in that A represents CO and B represents benzyl or 4-methylphenylmethyl and D represents benzoyl.

5. Use according to claim 4, characterized in that die compounds of formula (I) is selected from the group consisting of

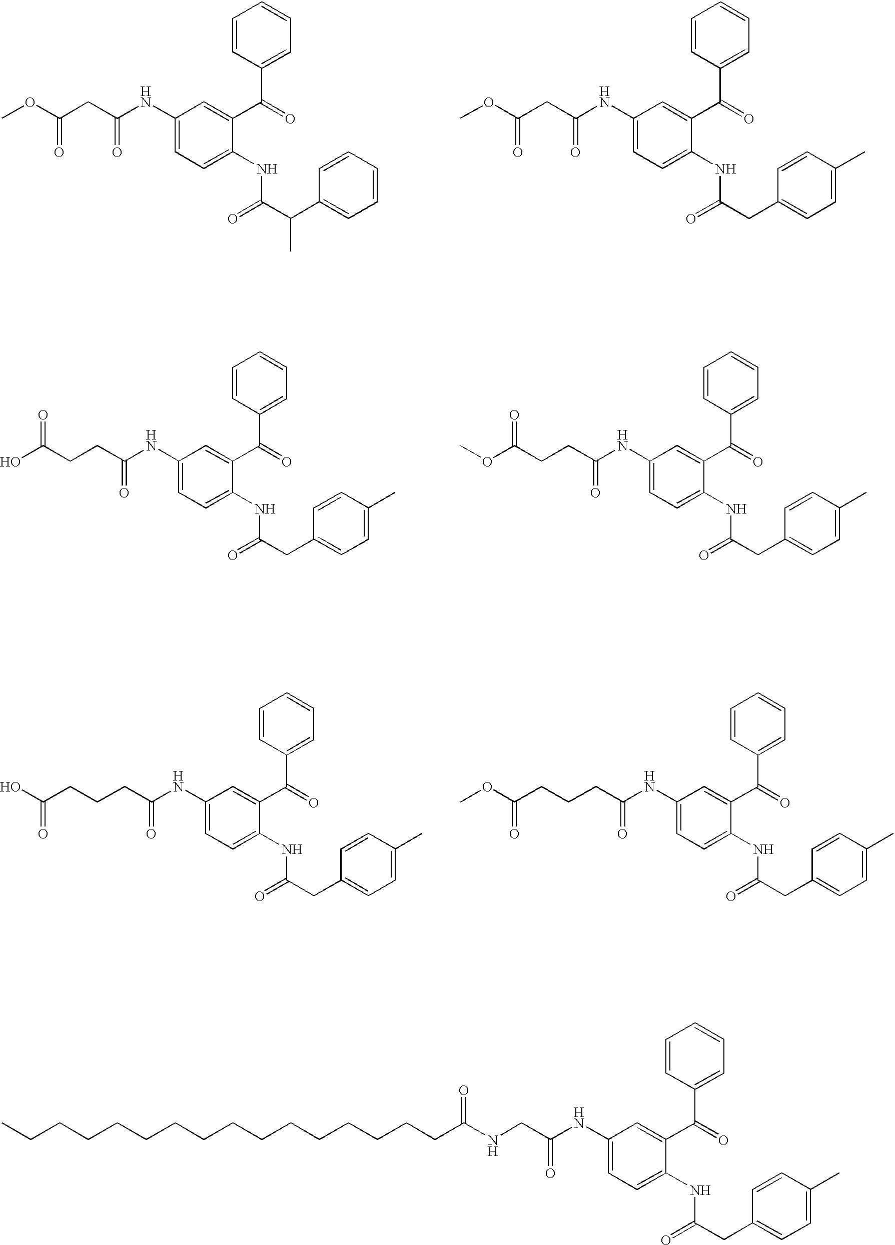

6. Use according to claim 1, characterized in that die compounds of formula (I) selected from the group consisting of N-[3-[3-benzoyl-4-[2-(4-methylphenyl)-acetyl]amino]-phenylamino]-4-phenyl cinnamic acid amide and N-[2-[3-benzoyl-4-[[2-(4-methylphenyl)acetyl]-amino]phenyl]-4-benzyloxy cinnamic acid amide.

7. Use according to one of claims 1 to 6 for the prophylactics and treatment of infections caused by parasites, especially unicellular parasites, namely pathogens of malaria, the sleeping sickness, the Chagas' disease, the toxoplasmosis, amoebic dysentery, leishmaniasis, trichomoniasis, pnemacystosis, balantidiosis, cryptosporidiosis, sarcocystosis, acanthamebiasis, naegleriasis, coccidiosis, giardiasis and lambliosis.

8. Pharmaceutical preparation for the therapeutic and prophylactic treatment of infectious processes, characterized in that the preparation contains an effective amount of at least one compound according to one of claims 1 to 6 together with a pharmaceutically acceptable carrier.

9. Pharmaceutical preparation according to claim 8, characterized in that the preparation contains a further pharmaceutical active agent.
